(19) United States
(12) Patent Application Publication
(10) Pub. No.: US 2022/0071976 A1
(43) Pub. Date: Mar. 10, 2022

(54) DOMPERIDONE COMPOSITIONS AND METHODS FOR TREATING DEPRESSION

(71) Applicant: CHASE THERAPEUTICS CORPORATION, Washington, DC (US)

(72) Inventors: Thomas N. CHASE, Washington, DC (US); Kathleen E CLARENCE-SMITH, Washington, DC (US)

(73) Assignee: CHASE THERAPEUTICS CORPORATION, Washington, DC (US)

(21) Appl. No.: 17/418,034

(22) PCT Filed: Dec. 12, 2019
(86) PCT No.: PCT/US2019/065973

§ 371 Date: Jun. 24, 2021

Related U.S. Application Data
(60) Provisional application No. 62/785,606, filed on Dec. 27, 2018.
(60) Provisional application No. 62/787,614, filed on Jan. 2, 2019.
(60) Provisional application No. 62/817,162, filed on Mar. 12, 2019.

(51) Int. Cl. A61K 31/4439; A61K 31/426

(52) U.S. Cl. CPC A61K 31/4439; A61K 31/426

(57) ABSTRACT

The present invention describes the combination of domperidone with pramipexole or a pharmaceutically acceptable salt or solvate thereof, useful for treating depressive disorders, including major depressive disorder.

### RELATED APPLICATIONS

This application claims the benefit of U.S. Provisional Patent Application Ser. No. 62/785,606, filed Dec. 27, 2018; and U.S. Provisional Patent Application Ser. No. 62/787,614, filed Jan. 2, 2019; and U.S. Provisional Patent Application Ser. No. 62/817,162, filed Mar. 12, 2019; the disclosures of which are herein incorporated in their entirety.

### FIELD OF THE INVENTION

The present invention pertains to the field of the treatment of depression.

### OBJECT OF THE INVENTION

The present invention includes pharmaceutical compositions and combinations, including fixed-dose combinations, and their use for the treatment of depression, in particular comprising the dopamine receptor antagonist 5-chloro-1-(1-[3-(2-oxo-2,3-dihydro-1H-benzo[d]imidazol-1-yl)propyl]piperidin-4-yl)-1H-benzo[d]imidazol-2(3H)-one (domperidone) or a pharmaceutically acceptable salt or solvate thereof for use for the treatment of depression in combination with (S)-6-propylamino-4,5,6,7-tetrahydro-1,3-benzothiazole-2-amine or a pharmaceutically acceptable salt or solvate thereof.

### Definitions

### “CGI”: Clinical Global Impression.

### “CNS”: Central Nervous System.

“IR”: Immediate Release of the active ingredient from a composition.
“ER”: Extended Release of the active ingredient from a composition.

### “GI”: Gastro-Intestinal.

### “AE(s)”: Adverse Effect(s).

“DSM-5”: Diagnostic and Statistical Manual of Mental Disorders, 5^{th} edition.

### “HAMD”: Hamilton Depression Rating Scale.

### “MADRS”: Montgomery and Asberg Depression Rating Scale.

### “MDD”: Major Depressive Disorder.

“MAOIs”: Monoamine oxidase inhibitors.

### “NIMH”: National Institute of Mental Health.

### “PD”: Parkinson's Disease.

“Persistent depressive disorder”: also called dysthymia.

### “PMDD”: Premenstrual Dysphoric Disorder.

“Effective domperidone dose per unit form”: This expression, as used herein, refers to a dose of domperidone or pharmaceutically acceptable salt or solvate thereof that is equivalent to from 2 mg to 120 mg of domperidone base, per unit form.
“Effective daily dose of domperidone”: This expression, as used herein, refers to a dose of domperidone or pharmaceutically acceptable salt or solvate thereof that is equivalent to from 4 mg to 120 mg of domperidone base daily.
“Pramipexole”: the (S)-6-propylamino-4,5,6,7-tetrahydro-1,3-benzothiazole-2-amine, as active principle including the free base and its pharmaceutically acceptable salts and solvates, unless otherwise specified.
“Effective daily dose of pramipexole” or “therapeutically effective dose of pramipexole”: a daily pramipexole dose equivalent to at least a pramipexole dihydrochloride monohydrate approved daily dose for the treatment of PD, this effective daily dose including low daily doses used during the titration period.
“Effective dose/unit form” or “effective dose per unit form” or “effective amount (or dose) per unit form”, in reference to pramipexole: a pramipexole amount per unit form equivalent to at least a pramipexole dihydrochloride monohydrate amount per unit form approved for the treatment of PD, this amount including low amounts per unit form used during the titration period.
“Salts or solvates thereof” or “salts and solvates thereof”, with reference to domperidone or to pramipexole: this expression indicates that any salt of said pramipexole or said domperidone may be solvated with a solvent, normally water. Unless otherwise specified, the terms designating the active principles “domperidone” and “pramipexole” include the free base, and salts and solvates thereof
“TDDS”: Transdermal Drug Delivery System.
“Maximum tolerated dose,” “maximal tolerated dose” or “MTD” refers to, and is defined as the highest dose of a drug or treatment that does not cause unacceptable side effects. For instance, the maximum tolerated dose is determined in clinical trials by testing increasing doses on different groups of people until the highest dose with acceptable side effects is found. The dose of the pramipexole may be higher than the maximum tolerated dose of pramipexole for the treatment of depression when administered alone. In particular, from 1.1 to 10 times higher than the maximal tolerated dose of pramipexole for the treatment of depression when administered alone, which includes, but is not limited to a dose from 1.5 to 10 times higher than the maximal tolerated dose of pramipexole for the treatment of depression when administered alone, a dose from 2.5 to 10 times higher than the maximal tolerated dose of pramipexole for the treatment of depression when administered alone, a dose from 3 to 10 times higher than the maximal tolerated dose of pramipexole for the treatment of depression when administered alone, a dose from 4 to 10 times higher than the maximal tolerated dose of pramipexole for the treatment of depression when administered alone, and a dose from 6 to 10 times higher than the maximal tolerated dose of pramipexole for the treatment of depression when administered alone.
The term “comprise,” “comprises,” “comprising” “include,” “includes,” and “including” are interchangeable and not intended to be limiting.
   It is to be further understood that where descriptions of various embodiments use the term “comprising,” those skilled in the art would understand that the present disclosure also contemplates such embodiments alternatively described using the language “consisting essentially of” or “consisting of”.
The transitional phrase “consisting essentially of” encompasses the specified materials or steps and those that do not materially affect the basic and novel characteristic(s) of the present invention. For instance, combinations and/or compositions consisting essentially of domperidone and 6-propylamino-4,5,6,7-tetrahydro-1,3-benzothiazole-2-amine. Such combinations or compositions of the present invention provide an unexpected increase in the therapeutic efficacy of pramipexole for treatment of depression (such as depressive disorders) in humans.
The transitional phrase “consisting of” excludes element(s), step(s), or material(s) not specified in the claim. For instance, the present invention provides combinations consisting of domperidone and 6-propylamino-4,5,6,7-tetrahydro-1,3-benzothiazole-2-amine, as well as compositions consisting of domperidone, 6-propylamino-4,5,6,7-tetrahydro-1,3-benzothiazole-2-amine, and at least one pharmaceutically acceptable excipient or carrier.

### BACKGROUND OF THE INVENTION

“Major depressive disorder” (MDD), also referred to as “depression” or “clinical depression”, is a common but serious mood disorder associated with a significant burden, affecting around 16% of the population in the US in their lifetime (reviewed in de Souza et al, 2015). Depression is one of the most common mental disorders in the U.S. Current research suggests that depression is caused by a combination of genetic, biological, environmental, and psychological factors.

The estimated costs of MDD are around 83 billion US Dollars annually, due to many psychosocial factors including loss of workdays (reviewed in de Souza et al, 2015). Estimates are that on average a depressed person loses 27.2 workdays per year (reviewed in de Souza et al, 2015). A significant part of the burden corresponds to unsuccessful treatments. Remission of depressive symptoms is achieved in only one-third of the MDD patients after the first antidepressant trial (reviewed in de Souza et al, 2015), and unsuccessful treatments contribute substantially to the observed suffering and social costs of MDD.

Signs and symptoms of depression typically include the following: persistent sad, anxious, or “empty” mood; feelings of hopelessness, or pessimism; irritability; feelings of guilt, worthlessness, or helplessness; loss of interest or pleasure in hobbies and activities; decreased energy or fatigue; moving or talking more slowly; feeling restless or having trouble sitting still; difficulty concentrating, remembering, or making decisions; difficulty sleeping, early-morning awakening, or oversleeping; appetite and/or weight changes; thoughts of death or suicide, or suicide attempts; aches or pains, headaches, cramps, or digestive problems without a clear physical cause and/or that do not ease even with treatment (NIMH, Health and Education, Mental Health Information as posted on the NIMH Web Site). Not everyone who is depressed experiences every symptom. Some people experience only a few symptoms while others may experience many. For a diagnosis of depression, signs and symptoms have to be present most of the day, nearly every day, for at least two weeks (DSM-5)

Depression, also herein referred to as “depressive disorders” can happen at any age (NIMH, Health and Education, Mental Health Information as posted on the NIMH Web Site), but often begins in adulthood. Depression is now recognized as occurring in children and adolescents, although it sometimes presents with more prominent irritability than low mood. Depression, especially in midlife or older adults, can co-occur with other serious medical illnesses, such as diabetes, cancer, heart disease, and Parkinson's disease. Risk factors include: personal or family history of depression; major life changes, trauma, or stress; certain physical illnesses and medications.

Some forms of depression, are slightly different, or develop under unique circumstances (NIMH, Health and Education, Mental Health Information as posted on the NIMH Web Site), such as:

Persistent depressive disorder (also called dysthymia) with early or late onset and with or without atypical features, is a depressed mood that lasts for at least two years. A person diagnosed with persistent depressive disorder may have episodes of major depression along with periods of less severe symptoms, but symptoms must last for two years to be considered persistent depressive disorder.
Peripartum (postpartum) depression, (PPD), situational depression, and atypical depression. The common feature of these depressive disorders is the presence of sad, empty, or irritable mood, accompanied by somatic and cognitive changes that significantly affect the individual's capacity to function. The difference among these disorders are issues of duration, timing or presumed etiology. See Depressive Disorders, Diagnostic and Statistical Manual of Mental Disorders, Fifth Edition, dsm.psychiatryonline.org/doi/10.1176/appi.books.9780890425596.dsm04.
Perinatal depression is much more serious than the “baby blues” (relatively mild depressive and anxiety symptoms that typically clear within two weeks after delivery) that many women experience after giving birth. Women with perinatal depression experience full-blown major depression during pregnancy or after delivery (postpartum depression). The feelings of extreme sadness, anxiety, and exhaustion that accompany perinatal depression may make it difficult for these new mothers to complete daily care activities for themselves and/or for their babies.
Psychotic depression occurs when a person has severe depression plus some form of psychosis, such as having disturbing false fixed beliefs (delusions) or hearing or seeing upsetting things that others cannot hear or see (hallucinations). The psychotic symptoms typically have a depressive “theme,” such as delusions of guilt, poverty, or illness.
Seasonal affective disorder is characterized by the onset of depression during the winter months, when there is less natural sunlight. This depression generally lifts during spring and summer. Winter depression, typically accompanied by social withdrawal, increased sleep, and weight gain, predictably returns every year in seasonal affective disorder.
Mood dysregulation disorder (diagnosed in children and adolescents; DSM-5).

### Premenstrual Dysphoric Disorder (PMDD; DSM-5).

Bipolar Disorder is different from depression, but it is included in this list because patients with bipolar disorder experience episodes of extremely low moods that meet the criteria for major depression (called “bipolar depression”). Bipolar disorder is a persistent, episodic and debilitating condition with an estimated lifetime prevalence of over 2.0%, including both types I (with mania) and II (with hypomania) (reviewed in Poon et al, 2015). Bipolar disorder is associated with recurring episodes of mania, hypomania, mixed manicdepressive states, or psychosis, as well as prominent major depression and dysthymia, as well as prevalent anxiety symptoms—all leading to high risks of potentially severe functional impairment, substance abuse, and high rates of suicide, accidents, and increased mortality from co-occurring medical illnesses—all despite use of available pharmacological and psychosocial treatments (Poon et al, 2015). The depressive components of the disorder have been especially difficult to treat successfully and they account for three-quarters of the several weeks of follow-up with treatment that include clinically significant residual morbidity (reviewed in Poon et al, 2015).

Other mood disorders encompassed, together with the aforementioned depressive disorders, within the term “depression” include Alzheimer's disease with depressed mood, depressed mood in Parkinson's disease, Lewy body disease, and other dementias, post-stroke depression, schizoaffective disorders, adjustment disorder with depressed mood, and drug- and alcohol-induced depressed mood.

Depression is usually initially treated with medications and psychotherapy. If the treatments do not reduce symptoms, electroconvulsive therapy and other brain stimulation therapies may help. Medications include the following (Mayo Clinic):

Selective serotonin reuptake inhibitors (SSRIs) such as fluoxetine (Prozac), paroxetine (Paxil, Pexeva), sertraline (Zoloft), citalopram (Celexa) and escitalopram (Lexapro).
Serotonin-norepinephrine reuptake inhibitors (SNRIs) such as duloxetine (Cymbalta), venlafaxine (Effexor XR), desvenlafaxine (Pristiq, Khedezla) and levomilnacipran (Fetzima).
Norepinephrine-dopamine reuptake inhibitors (NDRIs). Bupropion (Wellbutrin, Aplenzin, Forfivo XL) falls into this category.
Atypical antidepressants such as trazodone and mirtazapine (Remeron), vortioxetine (Brintellix) and vilazodone (Viibryd).
Tricyclic antidepressants (TCAs) such as imipramine (Tofranil), nortriptyline (Pamelor), amitriptyline, doxepin, trimipramine (Surmontil), desipramine (Norpramin) and protriptyline (Vivactil)—can be very effective, but tend to cause more-severe side effects than newer antidepressants. Tricyclics are therefore usually considered as second line therapy.
Monoamine oxidase inhibitors (MAOIs), such as tranylcypromine (Parnate), phenelzine (Nardil) and isocarboxazid (Marplan), may be prescribed, typically when other medications haven't worked. However, MAOIs are usually not first line antidepressant therapy, because they can have serious interactions with certain foods and some medications including birth control pills, decongestants and certain herbal supplements. Selegiline TDDS (Emsam), a newer MAOI, may cause fewer side effects than other MAOIs.

One disadvantage of all of the above antidepressant medications is that they typically take 2 to 4 weeks to start having an antidepressant effect.

(S)-6-propylamino-4,5,6,7-tetrahydro-1,3-benzothiazole-2-amine (pramipexole) is a synthetic aminothiazole derivative, described in U.S. Pat. No. 4,886,812, the content of which is incorporated herein by reference. It is a dopamine autoreceptor agonist (Schneider and Mierau 1987) that is approved for the treatment of the symptoms of Parkinson's disease (PD), in doses ranging from 0.375 mg/day to 4.5 mg/day, given in 3 equally divided doses (Mirapex® Prescribing Information May 2018) or in a single dose once a day (Mirapex ER® Prescribing Information, July 2016). Pramipexole is supplied in tablets for immediate release containing 0.125 mg, 0.25 mg, 0.5 mg, 1 mg and 1.5 mg of pramipexole dihydrochloride monohydrate; and in tablets for extended release containing 0.375 mg, 0.75 mg, 1.5 mg, 3 mg and 4.5 mg of pramipexole dihydrochloride monohydrate. It is structurally distinct from the ergot-derived drugs (e.g., bromocriptine and pergolide). Pramipexole is a dopamine D2 receptor agonist that is also pharmacologically unique in that it is a full agonist and has receptor selectivity for the dopamine D3 receptor subtype of the D2 subfamily of receptors. These properties may confer advantages in terms of both efficacy (full agonist with potential for greater therapeutic effects) and safety (receptor selectivity may reduce unwanted side effects) compared to currently available dopamine agonists (Piercey, 1998).

Literature reports that pramipexole was also found to be effective in the treatment of depressive symptoms in patients with PD, albeit with a small effect size. A 12-week, double-blind, placebo controlled trial in 296 PD patients was conducted with pramipexole (0.125 to 1.0 mg given three times per day). The primary endpoint was the Beck Depression Inventory scale (BDI). Results showed that BDI scores decreased by an adjusted 5.9 (SE 0.5) in the pramipexole group, and 4.0 (SE 0.5) in the placebo group. The difference between the two treatment groups was significant (p=0.01; Barone et al, 2010), although the magnitude of the effect size was small. In addition, other small, often open-label studies in which pramipexole was added on to antidepressant treatment (augmentation) also showed modest but significant efficacy in favor of pramipexole in non-PD patients with major depressive disorder (MDD; Cusin et al, 2013; Goldberg et al, 2004), including non-PD patients with treatment resistant depression (Hori and Kunigi, 2012; Pae, et al, 2013; Fawcett et al, 2016), or patients with depression associated with bipolar disorder (reviewed in Sienaert et al, 2013; Dell'Osso and Ketter, 2013; Tondo et al, 2014). However, Kleebatt et al (2017) in their review judged that clear proof of antidepressant efficacy had not been obtained for pramipexole, and attributed this to low levels of evidence, small sample sizes or discordant results. In all these reports, the dose of pramipexole remained within the range approved for the treatment of the motor symptoms of PD, even when the title of the publication mentions “high doses” of pramipexole (Fawcett et al, 2016). Since in most of these studies, efficacy appeared to be modest, higher doses of pramipexole were tested in a randomized, prospective, double-blind, placebo-controlled, fixed-dose study (Corrigan et al, 2000). A total of 174 eligible patients with a DSM-III-R diagnosis of major depression (single or recurrent episode, with or without melancholia and without psychotic features) were assigned to one of five treatment groups: placebo group, fluoxetine group (20 mg/day), or one of three pramipexole groups (0.375 mg/day; 1 mg/day; 5 mg/day). Patients received a 1-week placebo run-in, 8 weeks of treatment, and a 1-week post-study follow-up assessment (week 9). Efficacy was measured primarily by the change from baseline in the HAM-D (17-item version) total score, MADRS total score, and the CGI-Severity of Illness (SI) score. Results showed that the majority of patients in each treatment group completed the study (66-86%), with the exception of the pramipexole 5.0 mg group (42.4%). In the pramipexole 5.0 mg group, 57.6% of patients discontinued treatment prematurely, mainly because of adverse events (AEs), 76% of patients reported nausea, and 39% reported vomiting. At endpoint (week 8), the pramipexole 1.0 mg group and the fluoxetine group showed significantly better improvement over baseline than the placebo group on the HAMD (p=0.0076) and on the MADRS. The pramipexole 5.0 mg group had the best improvement at week 8 (−15.00), but p values were not available for this test against placebo because of the high drop-out rate.

Taken together, the results reported by Corrigan et al (2000) suggest that higher doses of pramipexole could be more effective, but doses higher than the approved doses cannot be used because of a high incidence of dose-limiting adverse events (AEs), notably constipation, nausea and vomiting but also non-G.I. adverse effects such as diaphoresis, light headedness and headache. Also, animal studies support the suggestion that high doses of pramipexole should be more effective for the treatment of depression. For example, high doses of pramipexole proved to be active in diverse tests of animal behavior simulating symptoms of depression, including Willner's Anhedonia Test (Willner et al., 1994), Fixed Interval Test, Forced Swimming Test, and REM Sleep Inhibition Test.

WO 2018/200387 discloses a combination of pramipexole with a 5HT3-antagonist antiemetic agent. Even though this antiemetic agent is efficacious for preventing and treating nausea and vomiting, its action on the intestinal disorders such as constipation caused by pramipexole is not satisfactory.

Thus, due to the pramipexole AEs, the problem of providing safe, chronic, effective treatment of a patient suffering from depression with pramipexole is not yet completely solved.

### SUMMARY OF THE INVENTION

It has been found that domperidone, by reducing or even abrogating the side effects of high doses of pramipexole (nausea, vomiting, acid reflux and constipation), enables the full antidepressant potential of pramipexole. In addition, contrary to the 5HT3-antagonists disclosed in WO 2018/200387 domperidone is able to counteract all the GI adverse effects of pramipexole.

Domperidone is a dopamine antagonist that is used for improving the delayed gastric emptying of functional origin with gastro-esophageal reflux and/or dyspepsia, for controlling nausea and vomiting of central or local origin, as an anti-emetic in patients receiving cytostatic and radiation therapy; and for facilitating radiological examination of the upper gastro-intestinal tract.

A study to investigate the effects of the D2-receptor agonist pramipexole (at a dose of 0.5 mg/day) with and without the co-administration of the peripherally acting D2-receptor antagonist domperidone (at a daily dose of 40 mg), on measures of alertness, autonomic and endocrine function, reports that a high enough concentration of the drug crosses the blood-brain barrier to partially antagonize some of the autonomic actions of pramipexole (Samuels et al. 2007). In particular, this report provides a cautionary note to the use of domperidone alongside pramipexole where the results of interest are those from pramipexole alone.

According to the present invention, by combining the dopamine D2-receptor agonist pramipexole and the dopamine-receptor antagonist domperidone, the central action of pramipexole is not antagonized and, surprisingly, pramipexole doses higher and even much higher than those recommended for the treatment of PD may be administered to a patient suffering from depression, with minimal adverse effects.

The present invention relates to increasing the therapeutic window for pramipexole, for the treatment of depressive disorders, including MDD, to safely enable its full antidepressant efficacy. In particular, the present invention relates to a combination of domperidone with pramipexole, to increase the therapeutic window of said pramipexole

Thus, the safe administration of pramipexole doses that are higher, and even much higher, than the maximum daily dose recommended for the relief of the symptoms of Parkinson's disease (such as motor symptoms), provides significant improvement to patients suffering from depressive disorders.

Alternatively, the safe administration of pramipexole doses that are higher, and even much higher than the maximum tolerated dose for the relief of symptoms of Parkinson's disease (such as motor symptoms), provides significant improvement to patients suffering from depressive disorders.

More particularly, it has been found that the combination, including fixed-dose combinations, of domperidone or a pharmaceutically acceptable salt or solvate thereof with pramipexole or a pharmaceutically acceptable salt or solvate thereof allows the administration of a therapeutic effective pramipexole dose that may be equivalent to from up to 10 times, in particular from 1.1 times to 4.7 times or from 1.1 times to 10 times higher than the aforementioned maximum dose of pramipexole dihydrochloride monohydrate recommended for the treatment of the symptoms of PD.

The combination of domperidone with pramipexole or a pharmaceutically acceptable salt or solvate thereof acts by enabling the full antidepressant efficacy of pramipexole, due to the high pramipexole doses that may be used in combination with said domperidone.

It has also been found that, in patients suffering from a depressive disorder, daily doses of pramipexole (in pramipexole dihydrochloride monohydrate) ranging from more than 4.5 mg to 45 mg/day, up to from 15 mg to 45 mg/day, normally from 14.5 mg to 25 mg or 15 mg to 25 mg, in combination with domperidone offer significant efficacy and a fast onset of action.

More particularly, it been found that, in the treatment of patients suffering from a depressive disorder, the use of domperidone in combination with daily doses of pramipexole or pharmaceutically acceptable salt or solvate thereof that are equivalent to from more than 4.5 mg to 22.5 mg/day and even from more than 6 mg to 22.5 mg or from 15 mg to 22.5 mg of pramipexole dihydrochloride monohydrate, also offer significant efficacy and a fast onset of action.

It has also been found that, by using domperidone, in combination with pramipexole or a pharmaceutically acceptable salt or solvate thereof, it is possible to treat a patient suffering from depression by maintaining an effective pramipexole or pharmaceutically acceptable salt or solvate thereof daily dose with minimal adverse effect.

Thus, the present invention provides a pharmaceutical combination or pharmaceutical composition comprising

(a) domperidone; and
(b) pramipexole or a pharmaceutically acceptable salt or solvate thereof.

In particular, the present invention provides a pharmaceutical combination or pharmaceutical composition comprising
(a) domperidone; and
(b) pramipexole or a pharmaceutically acceptable salt or solvate thereof, at a daily dose (in pramipexole dihydrochloride monohydrate) that is higher than the maximum dose of pramipexole dihydrochloride monohydrate recommended for the relief of the motor symptoms of PD,
   for use for the treatment of depressive disorders, including MDD.

Said pramipexole daily dose in said combination is up to 10 times, in particular from 1.1 times to 4.7 times, or from 1.1 times to 10 times higher than the maximum dose of pramipexole dihydrochloride monohydrate recommended for the relief of the motor symptoms of PD.

The present invention further provides the use of domperidone for enabling the full antidepressant efficacy of pramipexole in the treatment of MDD.

Moreover, the invention provides a method for treating a patient suffering from a depressive disorder, which comprises treating said patient with domperidone, in combination with pramipexole or a pharmaceutically acceptable salt or solvate thereof.

Moreover, the invention provides a method for treating a patient suffering from a depressive disorder, which comprises treating said patient with domperidone, in combination with pramipexole or a pharmaceutically acceptable salt or solvate thereof at a daily dose (in pramipexole dihydrochloride monohydrate) up to 10 times, in particular from 1.1 times to 4.7 times, or from 1.1 times to 10 times higher than the maximum daily dose recommended for the relief of the symptoms of Parkinson's disease such as motor symptoms (4.5 mg/day).

For carrying out the method (or for the use) according to the present invention, domperidone is formulated in a in dosage unit form comprising, as an active ingredient, said domperidone, in admixture with a pharmaceutical carrier or vehicle.

An advantageous domperidone Components (a) is selected from the group consisting of domperidone base, domperidone hydrochloride, domperidone maleate and domperidone succinate (1:1).

According to the present invention, domperidone is in a pharmaceutical composition comprising, as an active ingredient, said domperidone in an amount per unit form equivalent to from 2 mg to 120 mg of domperidone base, in admixture with a pharmaceutical carrier or vehicle.

The daily dose of the domperidone is from 4 to 120 mg and the pramipexole daily dose (in pramipexole dihydrochloride monohydrate), depending on the degree of gravity of the illness and the age and condition of the patient and including low doses for use during the titration period, will range from 0.375 mg to 45 mg, normally from 0.375 mg to 22.5 mg. For treating depression, said daily dose preferably is from more than 4.5 mg to 45 mg, from more than 6 mg to 45 mg, from 10 mg to 45 mg, from 13 mg to 45 mg, from 15 mg to 45 mg or from 15 mg to 25 mg, normally from more than 4.5 mg to 22.5 mg, in particular from more than 6 mg to 22.5 mg, from 10 mg to 22.5 mg, from 13 mg to 22.5 mg or from 15 mg to 22.5 mg.

According to an embodiment, for said use or said method, said domperidone, in an amount per unit form of from 2 mg to 120 mg, and said pramipexole or pharmaceutically acceptable salt or solvate thereof, in an amount equivalent to from 0.125 mg to 45 mg, from more than 4.5 mg to 22.5 mg or from more than 6 mg to 22.5 mg, normally from 15 mg to 25 mg, are each formulated in a pharmaceutical composition in admixture with a pharmaceutical carrier or vehicle and separately administered, concurrently or sequentially, to a patient in need of treatment with said combination, and in particular to a patient suffering from a depressive disorder, including MDD.

According to another embodiment, said domperidone and said pramipexole or pharmaceutically acceptable salt or solvate thereof are mixed together and formulated in a pharmaceutical composition (fixed-dose combination), in admixture with a pharmaceutical carrier. This pharmaceutical composition may be administered to a patient in need of said treatment, in particular to a patient suffering from a depressive disorder.

According to this embodiment, said domperidone, in an amount of from 2 mg to 120 mg, and said pramipexole or pharmaceutically acceptable salt or solvate thereof, in an amount equivalent to from 0.125 mg to 45 mg or from 15 mg to 25 mg of pramipexole dihydrochloride monohydrate are mixed together and formulated in a pharmaceutical composition (fixed-dose combination), in admixture with a pharmaceutical carrier or vehicle. In said composition, preferably, said pramipexole is present in said composition in an amount in a range selected from the group consisting of from more than 4.5 mg to 45 mg, from more than 6 mg to 45 mg, from 10 mg to 45 mg, from 13 mg to 45 mg, from 15 mg to 45 mg, from 7.5 mg to 25 mg, from 15 mg to 25 mg, from more than 4.5 mg to 22.5 mg or from more than 6 mg to 22.5 mg, from 7.5 mg to 22.5 mg, from 10 mg to 22.5 mg, from 13 mg to 22.5 mg, and from 15 mg to 22.5 mg of pramipexole dihydrochloride monohydrate. This pharmaceutical composition may be administered to a patient in need of said treatment, in particular to a patient suffering from a depressive disorder.

Thus, according to an aspect of this embodiment, the present invention provides a pharmaceutical composition for use in the treatment of depression comprising a pharmaceutically acceptable carrier or vehicle and a fixed dose combination of domperidone or a pharmaceutically acceptable salt or solvate thereof and pramipexole or a pharmaceutically acceptable salt or solvate thereof.

According to the invention, said domperidone may also be formulated in a pharmaceutical composition comprising said domperidone in an amount per unit form of from 2 mg to 120 mg, in admixture with a pharmaceutical carrier or vehicle, for use for preventing or curing the adverse effects of pramipexole daily doses that for some patients may be higher, and even much higher, than the maximum dose (4.5 mg/day) presently recommended for the relief of the motor symptoms of Parkinson's disease.

Domperidone may be used in the above pharmaceutical compositions in an amount per unit form within the above range, in particular in amount per unit form equivalent to a range selected from the group consisting of from 2 mg to 120 mg, from 2 mg to 100 mg, from 2 mg to 80 mg, from 2 mg to 60 mg, from 2 mg to 40 mg, from 2 mg to 30 mg, or from 2 mg to 20 mg, normally from 10 mg to 100 mg, from 10 mg to 80 mg, from 10 mg to 60 mg or from 10 mg to 40 mg, from 10 mg to 30 mg, or from 10 mg to 20 mg of domperidone base.

Using this compositions, domperidone is administered to a patient suffering from a depressive disorder, including MDD, at a daily dose equivalent to a range selected from the group consisting of from 4 mg to 120 mg, from 4 mg to 100 mg, from 4 mg to 80 mg, from 4 mg to 60 mg, from 4 mg to 40 mg, from 4 mg to 30 mg or from 4 mg to 20 mg, normally from 10 mg to 120 mg, from 10 mg to 100 mg, from 10 mg to 80 mg, from 10 mg to 60 mg, from 10 mg to 40 mg, from 10 mg to 30 mg or from 10 mg to 20 mg of domperidone base. Preferably, said domperidone is selected from the group consisting of domperidone base, domperidone hydrochloride, domperidone maleate and domperidone succinate (1:1).

For its use for the treatment of a depressive disorder, including MDD, in combination with domperidone, preferably in the above composition and daily dose, pramipexole is formulated in a pharmaceutical composition in dosage unit form comprising said pramipexole in an amount per IR- or ER-form (including low doses to be used during the titration period) equivalent to from 0.125 mg to 45 mg, advantageously from more than 4.5 mg to 45 mg, from more than 6 mg to 45 mg, from 7.5 mg to 45 mg, from 10 mg to 45 mg, from 13 mg to 45 mg, or from 15 mg to 45 mg normally from 7.5 mg to 25 mg or from 15 mg to 25 mg, of pramipexole dihydrochloride monohydrate.

In particular, for said use in combination with domperidone, pramipexole is formulated in a pharmaceutical composition in dosage unit form comprising said pramipexole an amount per IR-form (including low doses to be used during the titration period) equivalent to from 0.125 mg to 22.5 mg, advantageously from 1.5 mg to 22.5 mg, from more than 3 mg to 22.5 mg, from 5 mg to 22.5 mg, from 6.5 mg to 22.5 mg, or from 7.5 mg to 22.5 mg of pramipexole dihydrochloride monohydrate.

For said use in combination with domperidone, pramipexole, is formulated in a pharmaceutical composition in dosage unit form comprising said pramipexole, in an amount per ER-form (including low doses to be used during the titration period) equivalent to from 0.375 mg to 45 mg, from more than 4.5 mg to 45 mg, from 5 mg to 45 mg, from more than 6 mg to 45 mg, from 10 mg to 45 mg from 13 mg to 45 mg, or from 15 mg to 45 mg, normally from 15 mg to 25 mg, of pramipexole dihydrochloride monohydrate.

According to an embodiment, the dose of pramipexole or pharmaceutically acceptable salt or solvate thereof, in pramipexole dihydrochloride monohydrate, per IR- or ER-unit form, will range from 0.125 mg to 22.5 mg, advantageously in a range selected from the group consisting of from 1.6 mg 22.5 mg, from 1.8 mg to 22.5 mg, from 2.4 mg to 22.5 mg, from 3 mg to 22.5 mg, more advantageously from more than 4.5 mg to 22.5 mg, preferably from more than 6 mg to 22.5 mg, from 10 mg to 22.5 mg, from 13 mg to 22.5 mg, or from 15 mg to 22.5 mg.

Preferably, according to this embodiment, the dose per unit form of pramipexole or pharmaceutically acceptable salt or solvate thereof in an ER formulation, including slow-release compositions and transdermal therapeutic systems such as transdermal patches, for the treatment of a depressive disorder, including MDD, will range from an amount that is equivalent to from more than 4.5 mg to 45 mg, in particular from 4.8 mg to 45 mg or from more than 6 mg to 45 mg, normally from 14.5 mg to 25 mg or from 15 mg to 25 mg of pramipexole dihydrochloride monohydrate, depending on the tolerability (in combination with said domperidone).

The present invention also includes, but is not limited to, the following embodiments:

1. Domperidone for use for the treatment of depression in a patient in need of said treatment, in combination with an effective daily dose of (S)-6-propylamino-4,5,6,7-tetrahydro-1,3-benzothiazole-2-amine (pramipexole) or a pharmaceutically acceptable salt or solvate thereof.

2. Domperidone for use according to embodiment 1, wherein said effective daily dose of (S)-6-propylamino-4,5,6,7-tetrahydro-1,3-benzothiazole-2-amine (pramipexole) or a pharmaceutically acceptable salt or solvate thereof is equivalent to from 0.375 mg to 45 mg of pramipexole dihydrochloride monohydrate.

3. Domperidone, for use according to embodiment 1, wherein said effective daily dose of (S)-6-propylamino-4,5,6,7-tetrahydro-1,3-benzothiazole-2-amine (pramipexole) or a pharmaceutically acceptable salt or solvate thereof is equivalent to from 15 mg to 25 mg of pramipexole dihydrochloride monohydrate.

4. Domperidone for use according to embodiment 1, wherein, said domperidone is in a pharmaceutical composition comprising a pharmaceutical carrier and said domperidone, in an amount per unit form equivalent to from 2 mg to 120 mg of domperidone base, in combination with said pramipexole, also in a pharmaceutical composition comprising pramipexole or a pharmaceutically acceptable salt or solvate thereof, in an amount per unit form of from 0.125 mg to 45 mg of pramipexole dihydrochloride monohydrate in admixture with a pharmaceutical carrier or vehicle.

5. Domperidone for use according to embodiment 1, wherein said domperidone is in a pharmaceutical composition comprising a pharmaceutical carrier or vehicle and a fixed-dose combination of said domperidone or pharmaceutically acceptable salt or solvate thereof, in an amount equivalent to from 2 mg to 120 mg of domperidone base, and of pramipexole or pharmaceutically acceptable salt or solvate thereof, in an amount equivalent to from 0.125 mg to 45 mg of pramipexole dihydrochloride monohydrate.

6. Domperidone for use according to embodiment 4 or 5, wherein said pramipexole or pharmaceutically acceptable salt or solvate thereof is present in said composition in an amount equivalent to from 7.5 mg to 25 mg of pramipexole dihydrochloride monohydrate.

7. Domperidone for use according to any one of embodiments 4 to 6, wherein any composition is in dosage unit form.

8. A pharmaceutical composition for use in the treatment of depression comprising a pharmaceutically acceptable carrier or vehicle and a fixed dose combination of domperidone or a pharmaceutically acceptable salt or solvate thereof and pramipexole or a pharmaceutically acceptable salt or solvate thereof.

9. The composition of embodiment 8, wherein said domperidone or pharmaceutically acceptable salt or solvate thereof is present in an amount equivalent to from 2 mg to 120 mg of domperidone base and said pramipexole or pharmaceutically acceptable salt or solvate thereof is present in an amount equivalent to from 0.125 mg to 45 mg of pramipexole dihydrochloride monohydrate.

10. The composition of embodiment 8, wherein said carrier or vehicle is for an IR-formulation; and, in said fixed-dose combination,
said domperidone or pharmaceutically acceptable salt or solvate thereof is present in an amount equivalent to from 2 mg to 60 mg of domperidone base; and
said pramipexole or pharmaceutically acceptable salt or solvate thereof is present in an amount equivalent to from 7.5 mg to 22.5 mg or from 7.5 mg to 12.5 mg of pramipexole dihydrochloride monohydrate.

11. The composition of embodiment 8, wherein said carrier or vehicle is for an ER-formulation; and, in said fixed-dose combination,
said domperidone or pharmaceutically acceptable salt or solvate thereof is present in an amount equivalent to from 4 mg to 120 mg of domperidone base; and
said pramipexole or pharmaceutically acceptable salt or solvate thereof is present in an amount equivalent to from 15 mg to 45 mg or from 15 mg to 25 mg of pramipexole dihydrochloride monohydrate.

12. A pharmaceutical composition comprising
(a) domperidone or a pharmaceutically acceptable salt or solvate thereof, in an amount equivalent to from 2 mg to 120 mg of domperidone base; and
(b) pramipexole or a pharmaceutically acceptable salt or solvate thereof, in an amount equivalent to from 7.5 mg to 45 mg of pramipexole dihydrochloride monohydrate,
   in admixture with a pharmaceutical carrier or vehicle.

13. The composition according to any one of embodiments 8 to 12, which is in dosage unit form.

14. Use of domperidone for the preparation of a medicament for the treatment of depression in combination with pramipexole or a pharmaceutically acceptable salt or solvate thereof.

15. A method for the treatment of a patient suffering from depression, which comprises administering to said patient domperidone, at a daily dose (in domperidone base) of from 4 mg to 120 mg, in combination with pramipexole, at a daily dose (in pramipexole dihydrochloride monohydrate) of from 0.375 mg to 45 mg.

16. The method of embodiment 15 wherein said pramipexole daily dose (in pramipexole dihydrochloride monohydrate) is from 15 mg to 25 mg.

### DETAILED DESCRIPTION

The present invention concerns domperidone Component (a), as an adverse events inhibitor, in combination with pramipexole or a pharmaceutically acceptable salt or solvate thereof Component (b) at a daily dose equivalent to up to 10 times, in particular from 1.1 times to 4.7 times, or from 1.1 times to 10 times higher than the maximal pramipexole dihydrochloride monohydrate dose approved for the treatment of motor symptom of Parkinson's disease. Said combination, including fixed-dose combinations, is useful for the treatment of depressive disorders, including MDD. Said combination, including fixed-dose combinations, is also for use for the treatment of depressive disorders, including MDD.

The present invention also concerns domperidone Component (a), as a global inhibitor of the pramipexole dopaminergic peripheral adverse events, in combination with pramipexole or a pharmaceutically acceptable salt or solvate thereof Component (b) at a daily dose equivalent to from 1.1 to 10 times higher than the maximal tolerated dose of pramipexole for the treatment of depression when administered alone. Such a daily dose includes, but is not limited to a dose from 1.1 to 10 times higher than the maximal tolerated dose of pramipexole for the treatment of depression when administered alone, a dose from 1.5 to 10 times higher than the maximal tolerated dose of pramipexole for the treatment of depression when administered alone, a dose from 2.5 to 10 times higher than the maximal tolerated dose of pramipexole for the treatment of depression when administered alone, a dose from 3 to 10 times higher than the maximal tolerated dose of pramipexole for the treatment of depression when administered alone, a dose from 4 to 10 times higher than the maximal tolerated dose of pramipexole for the treatment of depression when administered alone, and a dose from 6 to 10 times higher than the maximal tolerated dose of pramipexole for the treatment of depression when administered alone.

In particular, the invention concerns, according to its aspects,
a method for treating depressive disorders, including MDD, by administration of domperidone to a patient in combination with an effective daily dose of pramipexole or pharmaceutically acceptable salt thereof;
domperidone Component (a), for use for the treatment of depressive disorders, including MDD, in a patient, in combination with, as Component (b), a daily dose of pramipexole or pharmaceutically acceptable salt or solvate thereof;
the use of domperidone for the preparation of a medicament for the treatment of depressive disorders, including MDD, in a patient, in combination (including fixed-dose combination) with an effective daily dose of pramipexole or a pharmaceutically acceptable salt thereof;
the use of domperidone for the preparation of a medicament for the treatment of a depressive disorder, including MDD, said medicament consisting of a pharmaceutical composition in dosage unit form comprising, as an active ingredient, said domperidone and, as another active ingredient, pramipexole or a pharmaceutically acceptable salt thereof; and
a method (or use) of domperidone as an inhibitor of the adverse effects of pramipexole in the treatment of a depressive disorder, including MDD.

The present invention also relates to a fixed-dose combination (a/b) comprising said domperidone Component (a) and said pramipexole or pharmaceutically acceptable salt or solvate thereof Component (b) in a pharmaceutical composition in dosage unit form in admixture with a pharmaceutically acceptable carrier or vehicle. This fixed-dose combination is useful and is for use for the treatment of depressive disorders, including MDD, in a patient.

Specifically, the invention provides a pharmaceutical composition for use in the treatment of depression comprising a pharmaceutically acceptable carrier or vehicle and a fixed dose combination of domperidone or a pharmaceutically acceptable salt or solvate thereof and pramipexole or a pharmaceutically acceptable salt or solvate thereof.

### The Domperidone Component (a)

Domperidone, 5-chloro-1-(1-[3-(2-oxo-2,3-dihydro-1H-benzo[d]imidazol-1-yl)propyl]piperidin-4-yl)-1H-benzo[d]imidazol-2(3H)-one

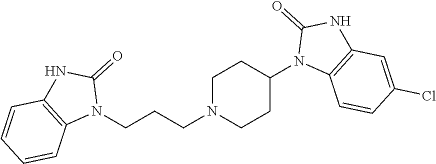 [SMILES: O=c1[nH]c2ccccc2n1CCCN1CCC(n2c(=O)[nH]c3cc(Cl)ccc32)CC1]

is a dopamine-2 receptor antagonist. It acts as an antiemetic and a prokinetic agent through its effects on the chemoreceptor trigger zone and motor function of the stomach and small intestine (Reddymasu et al. 2007), and is reputed not to enter the CNS (Brogden et al. 1982).

A study (Samuels et al. 2007) to investigate the effects of the D₂-receptor agonist pramipexole (at a dose of 0.5 mg/day) with and without the co-administration of the peripherally acting D₂-receptor antagonist domperidone (at a daily dose of 40 mg), on measures of alertness, autonomic and endocrine function, reports that a high enough concentration of the drug crosses the blood-brain barrier to partially antagonize some of the autonomic actions of pramipexole. In particular, this report provides a cautionary note to the use of domperidone alongside pramipexole where the results of interest are those from pramipexole alone.

As set forth above, domperidone is used for improving the delayed gastric emptying of functional origin with gastro-esophageal reflux and/or dyspepsia, for controlling nausea and vomiting of central or local origin, as an anti-emetic in patients receiving cytostatic and radiation therapy; and for facilitating radiological examination of the upper gastro-intestinal tract.

By combining domperidone with pramipexole according to the present invention, not only does the pramipexole effect size in depression become clinically significant by using pramipexole falling into the range considered safe and tolerable for human subjects by concurrently and safely interdicting the basic degenerative disease process in patients, but it is also possible to increase the pramipexole dose to a high degree.

The domperidone Component (a) is selected from the group consisting of domperidone base and pharmaceutically acceptable salt and solvates thereof.

Illustrative examples of pharmaceutically acceptable salts of domperidone include acid addition salts with, for example, hydrochloric acid, hydrobromic acid, sulfuric acid, nitric acid, phosphoric acid, acetic acid, propanoic acid, hydroxyacetic acid, 2-hydroxypropanoic acid, 2-oxopropanoic acid, propanedioic acid, butanedioic acid, (Z)-2-butenedioic acid (fumaric acid), (E)-2-butenedioic acid (maleic acid), 2-hydroxybutanedioic acid, 2,3-dihydroxybutanedioic acid, 2-hydroxy-1,2,3-propanetricarboxylic acid, benzoic acid, 3-phenyl-2-propenoic acid, .alpha.-hydroxybenzeneacetic acid, methanesulfonic acid, ethanesulfonic acid, benzenesulfonic acid, 4-methylbenzenesulfonic acid, cyclohexanesulfamic acid, 2-hydroxybenzoic acid, 4-amino-2-hydroxybenzoic acid. These salts are disclosed in U.S. Pat. No. 4,066,772, the contents of which are incorporated herewith in their entirety for reference. The solvation solvent is normally water.

A butanedioic acid addition salt, domperidone succinate 1:1, is particularly interesting because it could improve domperidone bioavailability, particularly in the fed state, and may reduce inter-patient variability, thus being advantageous for a pharmaceutical use (Bruni et al. 2013). Also, domperidone hydrochloride and domperidone maleate are particularly advantageous. Domperidone hydrochloride (Latha et al. 2012) and domperidone maleate (Shirisha et al. 2017) have been proposed in TDDS formulations.

In the method, use and combination, including fixed-dose combinations of the present invention, said domperidone Component (a) is present in an amount per unit form equivalent to from 2 mg to 120 mg of domperidone base; and is administered at a daily dose (in domperidone base) of from 4 mg to 120 mg.

For its administration to a patient suffering from depression, including pediatric patients, in combination with pramipexole Component (b), domperidone is formulated in a pharmaceutical composition in dosage unit form comprising, as an active ingredient, said domperidone, in admixture with a pharmaceutical carrier or vehicle.

An advantageous domperidone Components (a) is selected from the group consisting of domperidone base, domperidone hydrochloride, domperidone maleate and domperidone succinate (1:1).

According to the present invention, domperidone is in a pharmaceutical composition comprising, as an active ingredient, said domperidone in an amount per unit form equivalent to from 2 mg to 120 mg of domperidone base, in admixture with a pharmaceutical carrier or vehicle, and is administered at a daily dose equivalent to from 4 mg to 120 mg of domperidone base, in combination with a pramipexole daily dose equivalent to from 0.375 mg to 45 mg of pramipexole dihydrochloride monohydrate.

As set forth above, the aforementioned domperidone daily dose may vary according to the daily dose of pramipexole, administered therewith, as described in “The pramipexole Component (b)” section below.

Preferably, said domperidone is selected from the group consisting of domperidone base, domperidone hydrochloride, domperidone maleate and domperidone succinate (1:1).

A composition comprising domperidone as illustrated above, as Component (a), is administered to a patient suffering from a PMND in combination with pramipexole Component (b), also in a pharmaceutical composition comprising pramipexole or a pharmaceutically acceptable salt thereof in an amount equivalent to from 0.125 mg to 45 mg of pramipexole dihydrochloride monohydrate.

In said combination with pramipexole Component (b), domperidone Component (a) may also be in a fixed-dose combination (ab), wherein the amount of said domperidone Component (a) is equivalent to from 2 mg to 120 mg of domperidone base, and the amount of said pramipexole Component (b), in pramipexole dihydrochloride monohydrate is equivalent to from 0.125 mg to 45 mg of pramipexole dihydrochloride monohydrate.

A fixed-dose combination (ab) comprises, as an active ingredient, domperidone Component (a), in the above-illustrated amount-range; and, as a second active ingredient, pramipexole, in an effective amount as illustrated in “The pramipexole Component (b)” section below. Said fixed-dose combination normally is in a pharmaceutical composition to be administered to a patient suffering from a depressive disorder such as MDD.

Thus, the invention provides a pharmaceutical composition for the treatment of depression comprising a pharmaceutical carrier or vehicle and a fixed-dose combination of domperidone or a pharmaceutically acceptable salt or solvate thereof, and pramipexole or a pharmaceutically acceptable salt or solvate thereof.

According to an advantageous embodiment, the invention provides a pharmaceutical composition comprising
(a) domperidone or a pharmaceutically acceptable salt or solvate thereof, in an amount equivalent to from 2 mg to 120 mg of domperidone base; and
(b) pramipexole or a pharmaceutically acceptable salt or solvate thereof, in an amount equivalent to from 0.125 mg to 45 mg of pramipexole dihydrochloride monohydrate,
   in admixture with a pharmaceutical carrier or vehicle. Normally, said composition is in dosage unit form. Said composition is useful or for use for the treatment of depression.

According to the present invention, domperidone Component (a) may be present in the above pharmaceutical composition in an amount per unit form in the above range, in particular in amount per unit form equivalent to from 2 mg to 120 mg, from 2 mg to 100 mg, from 2 mg to 80 mg, from 2 mg to 60 mg, from 2 mg to 40 mg, from 2 mg to 30 mg, or from 2 mg to 20 mg, normally from 10 mg to 100 mg, from 10 mg to 80 mg, from 10 mg to 60 mg or from 10 mg to 40 mg, from 10 mg to 30 mg, or from 10 mg to 20 mg of domperidone base. Using this compositions, domperidone is administered to a patient suffering from a depressive disorder, including MDD, at a daily dose equivalent to from 4 mg to 120 mg, in particular equivalent to from 4 mg to 100 mg, from 4 mg to 80 mg, from 4 mg to 60 mg or from 4 mg to 40 mg, from 4 mg to 30 mg, or from 4 mg to 20 mg, normally from 10 mg to 100 mg, from 10 mg to 80 mg, from 10 mg to 60 mg, from 10 mg to 40 mg, from 10 mg to 30 mg, or from 10 mg to 20 mg of domperidone base.

Pramipexole is present in the above pharmaceutical composition in an amount per unit form in the above range, in particular in amount per unit form equivalent to from 0.125 mg to 45 mg, from 1.5 mg to 45 mg, from 3 mg to 45 mg, from more than 6 mg to 45 mg, from 7.5 mg to 45 mg, from 14.5 mg to 45 mg, from 15 mg to 45 mg, from more than 20 mg to 45 mg or from 20.25 mg to 45 mg, normally from 7.5 mg to 25 mg, from 15 mg to 25 mg, from more than 20 mg to 25 mg or from 20.25 mg to 25 mg.

Preferably, in said composition, said domperidone is selected from the group consisting of domperidone base, domperidone hydrochloride, domperidone maleate and domperidone succinate (1:1) and pramipexole Component (b) is present in an amount per unit form (in pramipexole dihydrochloride monohydrate) of from 7.5 mg to 25 mg, in particular in an amount of from 7.5 mg to 12.5 mg in an IR formulation or of from 15 mg to 25 mg in ER-formulation. Specific pramipexole Component (b) amounts per unit form are described in “The pramipexole Component (b)” section below.

The daily doses of pramipexole Component (b) in said fixed dose combinations (ab) are described below in “The pramipexole Component (b)” section.

### The Pramipexole Component (b)

A stated in the Definitions, the term “pramipexole” generally stands for (S)-6-propylamino-4,5,6,7-tetrahydro-1,3-benzothiazole-2-amine, as free base (pramipexole) or as pharmaceutically acceptable salts and solvates thereof, including pramipexole base and pramipexole dihydrochloride monohydrate, their doses per unit form and their daily doses being expressed as equivalents of pramipexole dihydrochloride monohydrate.

Pharmaceutically acceptable salts or solvates of pramipexole are also included in the present invention.

Illustrative examples of pharmaceutically acceptable salts or solvates of 6-propylamino-4,5,6,7-tetrahydro-1,3-benzothiazole-2-amine are derived from inorganic or organic acids such as hydrochloric acid, hydrobromic acid, sulfuric acid, sulfamic acid, phosphoric acid, nitric acid, acetic acid, propionic acid, stearic acid, glycolic acid, oxalic acid, succinic acid, lactic acid, maleic acid, hydroxymaleic acid, fumaric acid, malic acid, tartaric acid, citric acid, ascorbic acid, phenylacetic acid, glutamic acid, benzoic acid, salicylic acid, 2-acetoxybenzoic acid, methanesulfonic acid, ethanesulfonic acid, 2-hydroxyethanesulfonic (isethionic) acid, p-toluenesulfonic acid, 2-naphthalenesulfonic acid, 4-amino-benzenesulfonic (sulfanilic) acid, 2,6-naphthalenedisulfonic acid, 1,5-naphthalenedisulfonic acid, and pamoic (embonic) acid. The solvent of solvation is normally water.

Among pramipexole and pharmaceutically acceptable salts or solvates thereof, pramipexole dihydrochloride monohydrate, commercially available, is preferred, but pramipexole base may be preferably used for some circumstance, for example in transdermal therapeutic systems. Stable pharmaceutical compositions comprising pramipexole dihydrochloride monohydrate, disclosed in WO 2012/0140604 and in WO 2008/122638, the contents of each of which are incorporated herein by reference in their entirety; and sustained release compositions comprising pramipexole dihydrochloride monohydrate, disclosed in U.S. Pat. No. 8,399,016, the contents of which is incorporated herein by reference in its entirety, may be useful for use in combination with domperidone for the treatment of depressive disorders, including MDD.

As set forth in the definitions, the effective daily dose of pramipexole is a dose equivalent to at least the pramipexole dihydrochloride monohydrate approved daily dose for the treatment of PD. Said daily approved dose is from 0.375 mg to 4.5 mg. However, it is hereby specified that, according to the present invention, the combination of domperidone with said pramipexole allows the administration of pramipexole dihydrochloride monohydrate at daily doses as high as those approved for the treatment of Parkinson's disease without any adverse effect, but also allows the administration of pramipexole dihydrochloride monohydrate daily doses that are higher and also much higher than said approved doses. For example, the dose of pramipexole or a pharmaceutically acceptable salt thereof may be a daily dose equivalent to up to 10 times, from up to 4.7 times, or from 1.1 times to 10 times higher than the maximum recommended dose for the treatment of the symptoms of Parkinson's disease (such as motor symptoms).

The effective daily dose of pramipexole may also be a dose equivalent to at least a maximal tolerated dose of pramipexole dihydrochloride monohydrate used for the treatment symptoms of PD (such as motor symptoms). For example, the effective daily dose of pramipexole or a pharmaceutically acceptable salt thereof may be a daily dose equivalent to from 1.1 to 10 times higher than a dose equivalent to at least a maximal tolerated dose of pramipexole dihydrochloride monohydrate used for the treatment of depression when administered alone. Such an effective daily dose, includes but is not limited to, a dose equivalent to a dose from 1.1 to 10 times higher than the maximal tolerated dose of pramipexole dihydrochloride monohydrate used for the treatment of depression when administered alone, a dose from 1.5 to 10 times higher than the maximal tolerated dose of pramipexole dihydrochloride monohydrate used for the treatment of depression when administered alone, a dose equivalent to a dose from 2.5 to 10 times higher than the maximal tolerated dose of pramipexole dihydrochloride monohydrate used for the treatment of depression when administered alone, a dose equivalent to a dose from 3 to 10 times higher than the maximal tolerated dose of pramipexole dihydrochloride monohydrate used for the treatment of depression when administered alone, a dose equivalent to a dose from 4 to 10 times higher than the maximal tolerated dose of pramipexole dihydrochloride monohydrate used for the treatment of depression when administered alone, and a dose equivalent to a dose from 6 to 10 times higher than the maximal tolerated dose of pramipexole dihydrochloride monohydrate used for the treatment of depression when administered alone.

For the treatment of depressive disorders, including MDD, in combination with domperidone as described in “The Domperidone Component (a)” section above, pramipexole is formulated in a pharmaceutical composition comprising said pramipexole in an amount equivalent to from 0.125 mg to 45 mg of pramipexole dihydrochloride monohydrate, in admixture with a pharmaceutical carrier or vehicle. Said composition is administered to a patient in need of said treatment at daily dose of from 0.375 mg to 45 mg in combination with domperidone at a daily dose equivalent to from 4 mg to 120 mg of domperidone base.

In particular, in said combination with domperidone, pramipexole may be administered to a patient, including pediatric patients, suffering from a depressive disorder, including MDD, at a daily dose equivalent to from 0.375 mg to 45 mg, from more than 4.5 mg to 45 mg, from 5 mg to 45 mg, from more than 6 mg to 45 mg, from 10 mg to 45 mg, from 13 mg to 45 mg, from 15 mg to 45 mg, from 15 mg to 25 mg, from more than 20 mg to 25 or from 20.25 mg to 25 mg, normally from 0.375 mg to 22.5 mg, from more than 4.5 mg to 22.5 mg, from more than 6 mg to 22.5 mg, from 10 mg to 22.5 mg, from 13 mg to 22.5 mg, from 15 mg to 22.5 mg, from more than 20 mg to 22.5 mg or from 20.25 mg to 22.5 mg of pramipexole dihydrochloride monohydrate; depending on the tolerability (in combination with the domperidone).

Said pharmaceutical composition normally is in dosage unit form comprising said pramipexole in an amount per IR-form or ER-form (including low doses to be used during the titration period) equivalent to from 0.125 mg to 45 mg, from 7.5 mg to 45 mg, from more than 20 mg to 45 mg or from 20.125 mg to 45 mg, normally from 7.5 mg to 25 mg, from 15 mg to 25 mg, from more than 20 mg to 25 mg or from 20.25 mg to 25 mg. Advantageously, said amount per IR-form or per ER-form is from more than 4.5 mg to 45 mg, from more than 6 mg to 45 mg, from 10 mg to 45 mg, from 13 mg to 45 mg, or from 15 mg to 45 mg, normally from 15 mg to 25 mg or from 20.25 mg to 25 mg of pramipexole dihydrochloride monohydrate.

In particular, said pharmaceutical composition Component (b) comprises, as an active ingredient, pramipexole or a pharmaceutically acceptable salt thereof in an amount per unit form equivalent to from 0.125 mg to 22.5 mg, from 1.5 mg to 22.5 mg, from more than 3 mg to 22.5 mg, from 5 mg to 22.5 mg, from 6.5 mg to 22.5 mg, or from 7.5 mg to 22.5 mg, normally from 7.5 mg to 12.25 mg, from more than 10 mg to 12.25 mg or from 10.125 mg to 12.5 mg, of pramipexole dihydrochloride monohydrate, in an IR-formulation, or in an amount per unit form equivalent to from 0.375 mg to 45 mg, from more than 4.5 mg to 45 mg, from 5 mg to 45 mg, from more than 6 mg to 45 mg, from 10 mg to 45 mg from 13 mg to 45 mg, or from 15 mg to 45 mg, normally from 15 mg to 25 mg, from more than 20 mg to 25 mg, from 20.25 mg to 25 mg or from 20.25 mg to 22.5 mg of pramipexole dihydrochloride monohydrate in an ER-formulation. Said amounts per unit form include low amounts for use of said unit forms during the titration period.

In a preferred embodiment, in order to provide the treatment of a depressive disorder, including MDD, with high pramipexole doses, the invention provides a pharmaceutical composition in dosage unit form comprising, as an active ingredient, pramipexole or a pharmaceutically acceptable salts or solvates thereof in an amount per unit form equivalent to from 7.5 mg to 45 mg, from 12.5 mg to 45 mg from 15 mg to 45 mg, from 15 mg to 30 mg, from 15 mg to 25 mg or from 15 mg to 22.5 mg of pramipexole dihydrochloride monohydrate, in admixture with a pharmaceutical carrier or vehicle.

According to this preferred embodiment said pharmaceutical composition comprises, as an active ingredient, pramipexole or a pharmaceutically acceptable salts or solvates thereof in an amount per unit form (in pramipexole dihydrochloride monohydrate) equivalent to from 7.5 mg to 25 mg, in particular of from 7.5 mg to 12.5 mg in IR formulation or from 15 mg to 25 mg in ER-formulation.

Advantageously, according to this preferred embodiment said pharmaceutical composition may comprise, as an active ingredient, pramipexole or a pharmaceutically acceptable salts or solvates thereof in an amount per unit form (in pramipexole dihydrochloride monohydrate) equivalent to from 10.125 mg to 12.5 mg in IR formulation, or from 20.25 mg to 25 mg in ER-formulation.

As set forth above, domperidone, in combination with pramipexole or a pharmaceutically acceptable salt or solvate thereof, can be used to treat a patient suffering from a depressive disorder, including MDD, by maintaining a therapeutically effective pramipexole or pharmaceutically acceptable salt or solvate thereof daily dose with minimal adverse effect.

In order to provide concurrent administration of said domperidone or pharmaceutically acceptable salt or solvate thereof and of said pramipexole or pharmaceutically acceptable salt or solvate thereof, the invention also provides a fixed-dose combination comprising a pharmaceutical composition in dosage unit form comprising, as active ingredients, domperidone or pharmaceutically acceptable salts and solvates thereof and pramipexole or pharmaceutically acceptable salts and solvates thereof, in admixture with a pharmaceutical carrier or vehicle.

The domperidone/pramipexole fixed-dose combinations will be described in “The Pharmaceutical Compositions” section below.

First Aspect of the Invention

According to a first aspect, the present invention includes a method for safely treating a depressive disorder, including MDD, in patients suffering from a depressive disorder, with pramipexole by concurrently administering to said patients domperidone.

More particularly, the invention provides a method for treating a patient suffering from a depressive disorder, including MDD, which comprises administering to a patient in need of said treatment an effective dose of said domperidone in combination with an effective daily dose of pramipexole or a pharmaceutically acceptable salt thereof.

According to a preferred embodiment,

said domperidone is administered at a daily dose described in “The Domperidone Component (a)” section; and

said pramipexole or pharmaceutically acceptable salts or solvates thereof is administered at a daily dose as described above in “The pramipexole Component (b)” section.

In carrying out the method of the present invention, the daily dose of domperidone is at least as high as that recommended for improving the delayed gastric emptying of functional origin with gastro-esophageal reflux and/or dyspepsia, for controlling nausea and vomiting of central or local origin, as an anti-emetic in patients receiving cytostatic and radiation therapy; and for facilitating radiological examination of the upper gastro-intestinal tract. Said daily dose is equivalent to from 4 mg to 120 mg of domperidone base.

The pramipexole or a pharmaceutically acceptable salt or solvate thereof daily dose is equivalent to from 0.375 mg to 45 mg of pramipexole dihydrochloride monohydrate, including daily doses used during the titration period.

Preferably, in said combination, said domperidone is domperidone base, domperidone hydrochloride, domperidone maleate or domperidone succinate (1:1); and said pramipexole or a pharmaceutically acceptable salt or solvate thereof is pramipexole dihydrochloride monohydrate.

In particular, said domperidone in said combination is domperidone base at an effective daily dose of from 4 mg to 120 mg; and said pramipexole or pharmaceutically acceptable salt or solvate thereof in said combination is pramipexole dihydrochloride monohydrate, at an effective daily dose in a range selected from the group consisting of from more than 4.5 mg to 45 mg, from 5 mg to 45 mg, from more than 6 mg to 45 mg, from 10 mg to 45 mg, from 13 mg to 45 mg, or from 15 mg to 45 mg, normally from 15 mg to 25 mg, from more than 20 mg to 25 mg, from 20.25 mg to 25 mg, from 15 mg to 22.5 mg, from more than 20 mg to 22.5 mg and from 20.25 mg to 22.5 mg.

In carrying out the method of the present invention, said domperidone and said pramipexole or pharmaceutically acceptable salt or solvate thereof are each formulated in a pharmaceutical composition in dosage unit form comprising, respectively, said domperidone and said pramipexole or pharmaceutically acceptable salt or solvate thereof, each in admixture with a pharmaceutical carrier or vehicle.

Said domperidone and said pramipexole or pharmaceutically acceptable salt or solvate thereof may also be in a fixed-dose combination, co-formulated in a pharmaceutical composition in dosage unit form comprising an effective amount per unit form of said domperidone, and an effective amount per unit form of said pramipexole or pharmaceutically acceptable salts and solvates thereof, in admixture with a pharmaceutical carrier or vehicle. This fixed dose combination will be described herein below in the fourth aspect of the invention.

The doses per unit form of said domperidone and of said pramipexole are described above, respectively, in “The Domperidone Component (a)” and in “The pramipexole Component (b)” sections.

In particular, in said composition, said domperidone is present in an amount per unit form of from 2 mg to 120 mg.

In said composition, said pramipexole or pharmaceutically acceptable salt or solvate thereof is present in an amount per unit form equivalent to from 0.125 mg to 45 mg or from 0.125 mg to 22.5 mg of pramipexole dihydrochloride monohydrate.

In particular, said domperidone active ingredient of said pharmaceutical composition is selected from the group consisting of domperidone base, domperidone hydrochloride, domperidone maleate and domperidone succinate (1;1) in an amount per unit form equivalent to from 2 mg to 120 mg of domperidone base.

Said pramipexole dose per unit form consists of or includes an amount per unit form equivalent to a range selected from the group consisting of from 0.125 mg to 45 mg, from more than 4.5 mg to 45 mg, from 5 mg to 45 mg, from more than 6 mg to 45 mg, from 7.5 mg to 45 mg, from 10 mg to 45 mg, from 13 mg to 45 mg, and from 15 mg to 45 mg, normally from 7.5 mg to 25 mg from 15 mg to 25 mg, from more than 20 mg to 25 mg, from 20.25 mg to 25 mg, from 7.5 mg to 22.5 mg, from 15 mg to 22.5 mg, from more than 20 mg to 22.5 mg and from 20.25 mg to 22.5 mg. of pramipexole dihydrochloride monohydrate.

According to an embodiment, in the method of the present invention the domperidone is domperidone base; and pramipexole or a pharmaceutically acceptable salt or solvate thereof is administered at a daily dose that is equivalent to up to 10 times, in particular from 1.1 times to 4.7 times, or from 1.1 times to 10 times higher than the maximum recommended pramipexole dihydrochloride monohydrate dose approved for the relief of the symptoms of PD (such as motor symptoms).

According to another embodiment, in the method of the present invention the domperidone is domperidone base; and pramipexole or a pharmaceutically acceptable salt or solvate thereof is administered at a daily dose that is equivalent to a dose from 1.1 times to 10 times higher than a maximal tolerated dose of pramipexole dihydrochloride monohydrate dose for the treatment of depression when administered alone. Such a daily dose, includes but is not limited to, a daily dose that is equivalent to a dose from 1.1 to 10 times higher than the maximal tolerated dose of pramipexole dihydrochloride monohydrate dose for the treatment of depression when administered alone, a dose from 1.5 to 10 times higher than the maximal tolerated dose of pramipexole dihydrochloride monohydrate dose for the treatment of depression when administered alone, a daily dose that is equivalent to a dose from 2.5 to 10 times higher than the maximal tolerated dose of pramipexole dihydrochloride monohydrate dose for the treatment of depression when administered alone, a daily dose that is equivalent to a dose from 3 to 10 times higher than the maximal tolerated dose of pramipexole dihydrochloride monohydrate dose for the treatment of depression when administered alone, a daily dose that is equivalent to a dose from 4 to 10 times higher than the maximal tolerated dose of pramipexole dihydrochloride monohydrate dose for the treatment of depression when administered alone, and a daily dose that is equivalent to a dose from 6 to 10 times higher than the maximal tolerated dose of pramipexole dihydrochloride monohydrate dose for the treatment of depression when administered alone.

According to this embodiment, in said method (or use), said domperidone or pharmaceutically acceptable salt or solvate thereof, at the aforementioned effective daily dose, is administered to said patient, in combination with pramipexole or a pharmaceutically acceptable salt or solvate thereof administered to said patient at a daily dose equivalent to from more than 4.5 mg to 45 mg, from more than 4.5 mg to 25 mg, from more than 20 mg to 25 mg; or from more than 4.5 mg to 22.5 mg, normally from more than 6 mg to 22.5 mg, from 10 mg to 22.5 mg, from 13 mg to 22.5 mg or from 15 mg to 22.5 mg of pramipexole dihydrochloride monohydrate.

### Second Aspect of the Invention

According to a second aspect, the invention provides domperidone for use in combination with pramipexole or a pharmaceutically acceptable salt or solvate thereof for the treatment of a depressive disorder, including MDD, in a patient in need of said treatment.

In particular, this second aspect of the present invention provides
(a) domperidone, in combination with
(b) pramipexole or a pharmaceutically acceptable salt or solvate thereof, at a dose level (in pramipexole dihydrochloride monohydrate) that is up to 10 times, in particular from 1.1 times to 4.7 times, or from 1.1 times to 10 times higher than the maximum pramipexole dihydrochloride monohydrate daily dose recommended for the relief of the symptoms of Parkinson's disease (such as motor symptoms),
   for use for the treatment of depressive disorder, including MDD, in a patient.

Domperidone, as described in “The Domperidone Component (a)” section may be used, normally in a dosage unit form, according to this second aspect of the invention.

In particular, this second aspect of the present invention provides domperidone, in an amount per unit form equivalent to from 2 mg to 120 mg of domperidone base, for use in combination with a daily dose of said pramipexole or pharmaceutically acceptable salt or solvate thereof (including low doses used in the titration period) equivalent to from 0.375 mg to 45 mg, normally from 15 mg to 25 mg or from 15 mg to 22.5 mg of pramipexole dihydrochloride monohydrate, for the treatment of a depressive disorder, including MDD, in a patient in need of said treatment.

For the use according to this second aspect of present invention, the daily dose of said domperidone is at least as high as that indicated for improving the delayed gastric emptying of functional origin with gastro-esophageal reflux and/or dyspepsia, for controlling nausea and vomiting of central or local origin, as an anti-emetic in patients receiving cytostatic and radiation therapy; and for facilitating radiological examination of the upper gastro-intestinal tract. Said daily dose will range from 2 mg to 120 mg.

For its use for the treatment of a depressive disorder, including MDD, according to the present invention, domperidone, at the aforementioned effective daily dose, as described in “The Domperidone Component (a)” section, is administered to a patient in need of said treatment in combination with pramipexole at the aforementioned effective daily dose, as described in “The pramipexole Component (b)” section.

Normally, for its use according to this second aspect, the invention provides domperidone Component (a), in a pharmaceutical composition comprising, as an active ingredient, said domperidone, in admixture with a pharmaceutical carrier or vehicle, for use for the treatment of a depressive disorder, including MDD, in a patient, in combination with pramipexole or a pharmaceutically acceptable salt or solvate thereof Component (b), also in a pharmaceutical composition, in admixture with a pharmaceutical carrier or vehicle, to be administered to said patient at a daily dose that is equivalent to from up to 10 times, from up to 4.7 times, or from 1.1 times to 10 times higher than the maximum pramipexole dihydrochloride monohydrate daily dose recommended for the relief of the motor symptoms of Parkinson's disease (such a motor symptoms).

As another use according to this second aspect, the invention provides domperidone Component (a), in a pharmaceutical composition comprising, as an active ingredient, said domperidone, in admixture with a pharmaceutical carrier or vehicle, for the use of the treatment of a depressive disorder, including MDD, in a patient, in combination with pramipexole or a pharmaceutically acceptable salt or solvate thereof Component (b), also in a pharmaceutical composition, in admixture with a pharmaceutical carrier or vehicle, to be administered to said patient at a daily dose that is equivalent to a dose from 1.1 times to 10 times higher than a maximal tolerated dose of pramipexole dihydrochloride monohydrate dose for the treatment of depression when administered alone. Such a daily dose, includes but is not limited to, a daily dose that is equivalent to a dose from 1.1 to 10 times higher than the maximal tolerated dose of pramipexole dihydrochloride monohydrate dose for the treatment of depression when administered alone, a daily dose that is equivalent to a dose from 1.5 to 10 times higher than the maximal tolerated dose of pramipexole dihydrochloride monohydrate dose for the treatment of depression when administered alone, a daily dose that is equivalent to a dose from 2.5 to 10 times higher than the maximal tolerated dose of pramipexole dihydrochloride monohydrate dose for the treatment of depression when administered alone, a daily dose that is equivalent to a dose from 3 to 10 times higher than the maximal tolerated dose of pramipexole dihydrochloride monohydrate dose for the treatment of depression when administered alone, a daily dose that is equivalent to a dose from 4 to 10 times higher than the maximal tolerated dose of pramipexole dihydrochloride monohydrate dose for the treatment of depression when administered alone, and a daily dose that is equivalent to a dose from 6 to 10 times higher than the maximal tolerated dose of pramipexole dihydrochloride monohydrate dose for the treatment of depression when administered alone.

Said pharmaceutical composition in dosage unit form comprises said domperidone Component (a), in an amount equivalent to from 2 mg to 120 mg of domperidone base, in admixture with a pharmaceutical carrier or vehicle, and is for use for the treatment of a depressive disorder, including MDD, in a patient, in combination with pramipexole or a pharmaceutically acceptable salt or solvate thereof Component (b), in doses, in pramipexole dihydrochloride monohydrate, from up to 10 times, from up to 4.7 times, or from 1.1 times to 10 times higher than the daily dose approved for the relief of the symptoms of PD (such as motor symptoms).

Said pharmaceutical composition in dosage unit form comprises said domperidone Component (a), in an amount equivalent to from 2 mg to 120 mg of domperidone base, in admixture with a pharmaceutical carrier or vehicle, and is for use for the treatment of a depressive disorder, including MDD, in a patient, in combination with pramipexole or a pharmaceutically acceptable salt or solvate thereof Component (b), in doses, in pramipexole dihydrochloride monohydrate, from 1.1 times to 10 times higher than a maximal tolerated dose of pramipexole dihydrochloride monohydrate dose for the treatment of depression when administered alone. Such a dose, includes but is not limited to, a daily dose that is equivalent to a dose from 1.1 to 10 times higher than the maximal tolerated dose of pramipexole dihydrochloride monohydrate dose for the treatment of depression when administered alone, a daily dose that is equivalent to a dose from 1.5 to 10 times higher than the maximal tolerated dose of pramipexole dihydrochloride monohydrate dose for the treatment of depression when administered alone, a daily dose that is equivalent to a dose from 2.5 to 10 times higher than the maximal tolerated dose of pramipexole dihydrochloride monohydrate dose for the treatment of depression when administered alone, a daily dose that is equivalent to a dose from 3 to 10 times higher than the maximal tolerated dose of pramipexole dihydrochloride monohydrate dose for the treatment of depression when administered alone, a daily dose that is equivalent to a dose from 4 to 10 times higher than the maximal tolerated dose of pramipexole dihydrochloride monohydrate dose for the treatment of depression when administered alone, and a daily dose that is equivalent to a dose from 6 to 10 times higher than the maximal tolerated dose of pramipexole dihydrochloride monohydrate dose for the treatment of depression when administered alone.

According to an embodiment, said domperidone, in an amount per unit form equivalent to from 2 mg to 120 mg of domperidone base, is for use in the treatment of a depressive disorder, including MDD, in a patient in combination with a pramipexole, also in a pharmaceutical composition in dosage unit form comprising said pramipexole in an amount per unit form equivalent to from 0.125 mg to 45 mg, from more than 4.5 mg to 45 mg, from more than 6 mg to 45 mg, from 7.5 mg to 45 mg, from 10 mg to 45 mg, from 13 mg to 45 mg, or from 15 mg to 45 mg, normally from 7.5 mg to 25 mg or from 7.5 mg to 22.5 mg of pramipexole dihydrochloride monohydrate, to be administered to said patient at a daily dose equivalent to from 0.375 mg to 45 mg, from more than 4.5 mg to 45 mg, from more than 6 mg to 45 mg, from 10 mg to 45 mg, from 13 mg to 45 mg, or from 15 mg to 45 mg, normally from 15 mg to 25 mg, from more than 20 mg to 25 mg, from 20.25 mf to 25 mg or from 15 mg to 22.5 mg of pramipexole dihydrochloride monohydrate.

In particular, said domperidone is selected from the group consisting of domperidone base, domperidone hydrochloride, domperidone maleate and domperidone succinate (1;1), in an amount per unit form equivalent to from 2 mg to 120 mg of domperidone base.

Pramipexole, in said combination, may be formulated in a pharmaceutical composition in IR- or ER-form, in an amount per unit form as described in “The pramipexole Component (b)” section and administered twice to three times per day in an IR-formulation or once a day in an ER-formulation, at the aforementioned daily doses, in combination with domperidone.

### Third Aspect of the Invention

According to this third aspect, the present invention provides the use of domperidone for the preparation of a medicament for the treatment of a depressive disorder, including MDD, in a patient in need of said treatment, in combination with pramipexole or a pharmaceutically acceptable salt or solvate thereof.

This third aspect of the invention provides the use of said domperidone Component (a), for the preparation of a medicament consisting of a pharmaceutical composition comprising, as an active ingredient, said domperidone, in an amount equivalent to from 2 mg to 120 mg of domperidone base, in admixture with a pharmaceutical carrier or vehicle, for the treatment of a depressive disorder, including MDD, in a patient, in combination with pramipexole or a pharmaceutically acceptable salt or solvate thereof Component (b), at a daily dose, (in pramipexole dihydrochloride monohydrate) up to 10 times, in particular from 1.1 times to 4.7 times, or from 1.1 times to 10 times higher than the daily dose approved for the relief of the symptoms of PD (such as motor symptoms).

This third aspect of the invention also provides the use of said domperidone Component (a), for the preparation of a medicament consisting of a pharmaceutical composition comprising, as an active ingredient, said domperidone, in an amount equivalent to from 2 mg to 120 mg of domperidone base, in admixture with a pharmaceutical carrier or vehicle, for the treatment of a depressive disorder, including MDD, in a patient, in combination with pramipexole or a pharmaceutically acceptable salt or solvate thereof Component (b), at a daily dose, (in pramipexole dihydrochloride monohydrate) from 1.1 times to 10 times higher than a maximal tolerated dose of pramipexole dihydrochloride monohydrate dose for the treatment of depression when administered alone. Such a daily dose, includes but is not limited to, a daily dose that is equivalent to a daily dose that is equivalent to a dose from 1.1 to 10 times higher than the maximal tolerated dose of pramipexole dihydrochloride monohydrate dose for the treatment of depression when administered alone, a dose from 1.5 to 10 times higher than the maximal tolerated dose of pramipexole dihydrochloride monohydrate dose for the treatment of depression when administered alone, a daily dose that is equivalent to a dose from 2.5 to 10 times higher than the maximal tolerated dose of pramipexole dihydrochloride monohydrate dose for the treatment of depression when administered alone, a daily dose that is equivalent to a dose from 3 to 10 times higher than the maximal tolerated dose of pramipexole dihydrochloride monohydrate dose for the treatment of depression when administered alone, a daily dose that is equivalent to a dose from 4 to 10 times higher than the maximal tolerated dose of pramipexole dihydrochloride monohydrate dose for the treatment of depression when administered alone, and a daily dose that is equivalent to a dose from 6 to 10 times higher than the maximal tolerated dose of pramipexole dihydrochloride monohydrate dose for the treatment of depression when administered alone.

For this use, said domperidone is formulated in a medicament consisting of or comprising a pharmaceutical composition in dosage unit form to be administered to a patient suffering from a depressive disorder, including MDD, in combination with pramipexole or a pharmaceutically acceptable salt or solvate thereof, also in a pharmaceutical composition in dosage unit form.

The above medicament consisting of or comprising combinations of pharmaceutical compositions, for use for the treatment of depressive disorder, including MMD, in a patient, normally are in dosage unit form, in an IR or ER formulation, and each of said compositions may comprise
(a) said domperidone, in admixture with a pharmaceutical carrier or vehicle, and/or
(b) said pramipexole or pharmaceutically acceptable salt or solvate thereof, in admixture with a pharmaceutical carrier or vehicle, or
(a/b) both said domperidone and said pramipexole or pharmaceutically acceptable salt or solvate thereof, in a fixed-dose combination comprising said domperidone and said pramipexole or pharmaceutically acceptable salt or solvate thereof in admixture with a pharmaceutical carrier or vehicle.

These medicaments consisting of or comprising combinations of pharmaceutical compositions, each pharmaceutical composition comprising Component (a) and/or Component (b) or the (a/b)-fixed-dose combination, are useful and for use for the treatment of depressive disorder, including MMD, in a patient.

Thus, this third aspect of the invention provides the use of domperidone for the preparation of a pharmaceutical composition in dosage unit form comprising, as an active ingredient in admixture with a pharmaceutical carrier or vehicle, said domperidone in an amount per unit form equivalent to from 2 mg to 120 mg of domperidone base (for use) for the treatment of a depressive disorder, including MDD, in a patient in need of said treatment, in combination with pramipexole, also in a pharmaceutical composition in dosage unit form comprising, in admixture with a pharmaceutical carrier or vehicle, said pramipexole in an amount per unit form equivalent to from 0.125 mg to 45 mg-of pramipexole dihydrochloride monohydrate, to be administered to said patient at a daily dose equivalent to from 0.375 mg to 45 mg of pramipexole dihydrochloride monohydrate.

As set forth above, the above pramipexole dose-range (daily and per unit form) includes low doses to be used during the titration period.

In particular, in said pharmaceutical combination,
(a) said domperidone is present in said composition in an amount per unit form of equivalent to from 2 mg to 120 mg of domperidone base; and
(b) said pramipexole Component (b) is present in said composition in an amount per unit form equivalent to from 0.125 mg to 45 mg, from more than 4.5 mg to 45 mg, from more than 6 mg to 45 mg, from 7.5 mg to 45 mg, from 10 mg to 45 mg, from 13 mg to 45 mg, or from 15 mg to 45 mg, normally from 7.5 mg 25 mg, from 15 mg to 25 mg, from more than 20 mg to 25 mg or from 20.25 mg to 25 mg, of pramipexole dihydrochloride monohydrate, to be administered at a daily dose equivalent to from 0.375 mg to 45 mg, preferably from more than 4.5 mg to 45 mg, from more than 6 mg to 45 mg, from 10 mg to 45 mg, from 13 mg to 45 mg, or from 15 mg to 45 mg, normally from 15 mg to 25 mg, from more than 20 mg to 25 mg or from 20.25 mg to 25 mg of pramipexole dihydrochloride monohydrate.

According to an embodiment of this third aspect, the invention provides a medicament consisting of or comprising a pharmaceutical combination comprising, as Components,
Component (a) domperidone, in a pharmaceutical composition in dosage unit form comprising, as an active ingredient, said domperidone, in an amount per unit form equivalent to from 2 mg to 120 mg of domperidone base, in admixture with a pharmaceutical carrier or vehicle; and
Component (b) pramipexole or a pharmaceutically acceptable salt or solvate thereof, in a pharmaceutical composition in dosage unit form comprising, as an active ingredient, said pramipexole or a pharmaceutically acceptable salt or solvate thereof, in an amount per unit form equivalent to from 0.125 mg to 45 mg or from 0.125 mg to 22.5 mg of pramipexole dihydrochloride monohydrate, in admixture with a pharmaceutical carrier or vehicle.

The pharmaceutical combination according to this third aspect of the invention may be administered to patients suffering from a depressive disorder, including MDD, with the intent of finding and adopting a safe and effective pramipexole daily dose with higher therapeutic efficacy than is used or known in the art, for the heretofore unachieved treatment or alleviation of symptoms of said depressive disorder, including MDD, in each patient. Normally, Component (a) and Component (b) are concurrently or sequentially administered to said patient suffering from a depressive disorder, including MDD.

Preferably, said Domperidone is selected from the group consisting of domperidone base, domperidone hydrochloride, domperidone maleate and domperidone succinate (1:1), in an amount per unit form equivalent to from 2 mg to 120 mg of domperidone base.

In the pharmaceutical composition Component (b), pramipexole or pharmaceutically acceptable salt or solvate thereof, is in an amount per unit form equivalent to from 0.125 mg to 45 mg, in particular from more than 4.5 mg to 45 mg, from more than 6 mg to 45 mg, from 7.5 mg to 45 mg, from 10 mg to 45 mg, from 13 mg to 45 mg, or from 15 mg to 45 mg, normally from 7.5 mg to 25 mg, from more than 20 mg to 25 mg or from 20.25 mg to 25 mg of pramipexole dihydrochloride monohydrate.

In particular, the dose of pramipexole or pharmaceutically acceptable salt or solvate thereof per IR-unit form, for the treatment of a depressive disorder, including MDD, will range from an amount equivalent to from 0.125 mg to 22.5 mg, from 1.5 to 22.5 mg, from more than 3 mg to 22.5 mg, from 5 mg to 22.5 mg, from 6.5 mg to 22.5 mg, from 7.5 mg to 22.5 mg or from 10 mg to 22.5 mg, normally from 7.5 mg to 12.5 mg, from more than 10 mg to 12.5 mg or from 10.125 mg to 12.5 mg of pramipexole dihydrochloride monohydrate, depending on safety and tolerability (in combination with domperidone).

The dose per unit form of pramipexole or pharmaceutically acceptable salt or solvate thereof in an ER formulation, including slow-release compositions and transdermal therapeutic systems such as transdermal patches, for the treatment of a depressive disorder, including MDD, will range from an amount that is equivalent to from 0.375 mg to 45 mg, from 5 mg to 45 mg, from more than 6 mg to 45 mg, from 10 mg to 45 mg from 13 mg to 45 mg, or from 15 mg to 45 mg, normally from 15 mg to 25 mg, from more than 20 mg to 25 mg or from 20.25 mg to 25 mg of pramipexole dihydrochloride monohydrate, depending on the tolerability (in combination with said domperidone).

According to an embodiment, in the pharmaceutical composition Component (b), pramipexole or pharmaceutically acceptable salt or solvate thereof, is in an amount per unit form equivalent to from 0.125 mg to 22.5 mg, in particular from 0.125 mg to less than 1.6 mg, from 1.6 mg to 22.5 mg, from more than 4.5 mg to 22.5 mg or from more than 6 mg to 22.5 mg or from 7.5 mg to 22.5 mg of pramipexole dihydrochloride monohydrate.

In particular, the dose of pramipexole or pharmaceutically acceptable salt or solvate thereof per IR-unit form, for the treatment of a depressive disorder, including MDD, will be in an amount-range (in pramipexole dihydrochloride monohydrate) selected from the group consisting of from 0.125 mg to 22.5 mg, normally from 7.5 mg to 12.5 mg, from more than 10 mg to 12.5 and from 10.125 mg to 12.5 mg of pramipexole dihydrochloride monohydrate; or from 1.6 mg to 10.5 mg, from 1.8 to 10.5 mg, from 2.4 mg to 10.5 mg from 3 mg to 10.5 mg and from 7.5 mg to 10.5 mg, depending on safety and tolerability (in combination with domperidone).

The dose per unit form of pramipexole or pharmaceutically acceptable salt or solvate thereof in an ER formulation, including slow-release compositions and transdermal therapeutic systems such as transdermal patches, for the treatment of a depressive disorder, including MDD, according to this embodiment, will be in an amount-range (in pramipexole dihydrochloride monohydrate) selected from the group consisting of from 0.125 mg to 45 mg, normally from 15 mg to 25 mg, from more than 20 mg to 25 mg and from 20.25 mg to 25 mg of pramipexole dihydrochloride monohydrate; or from more than 4.5 mg to 22.5 mg, from 4.8 mg to 22.5 mg, from more than 6 mg to 22.5 mg, from 15 mg to 22.5 mg, from more than 20 mg to 22.5 mg or from 20.25 mg to 22.5 mg, depending on the tolerability (in combination with said domperidone).

In the case of separate (concurrent or sequential) administration of said domperidone, in an effective amount per unit form, and of said pramipexole, in an effective amount per unit form, each of them can be packaged in a kit comprising said domperidone, in admixture with a pharmaceutical carrier or vehicle, in a container; and said pramipexole, in admixture with a pharmaceutical carrier or vehicle, in another, separate container.

For the intended use in the treatment of a depressive disorder, including MDD, in combination with pramipexole, domperidone is formulated in a pharmaceutical composition, wherein said Domperidone is in admixture with a pharmaceutical carrier or vehicle.

For their concurrent administration for the treatment of depressive disorder, including MDD, said Domperidone and said pramipexole may also be formulated together and with a pharmaceutical carrier or vehicle, in a pharmaceutical composition (fixed-dose combination).

### Fourth Aspect of the Invention

A fourth aspect of the present invention provides
a pharmaceutical composition for use in the treatment of depression comprising a pharmaceutical carrier or vehicle and a fixed-dose combination of domperidone or a pharmaceutically acceptable salt or solvate thereof and pramipexole or a pharmaceutically acceptable salt or solvate thereof;
the use of domperidone for the manufacture of a medicament for the treatment of a depressive disorder, including MDD, said medicament consisting of or comprising a pharmaceutical composition in dosage unit form which comprises, as an active ingredient, said domperidone, in an effective amount per unit form, and, as a second active ingredient, pramipexole or a pharmaceutically acceptable salt thereof, in an effective amount per unit form, in admixture with a pharmaceutical carrier or vehicle;
the use of domperidone for the manufacture of a medicament for the treatment of a depressive disorder, including MDD, as a fixed-dose combination consisting of domperidone or a pharmaceutically acceptable salt or solvate thereof and pramipexole or a pharmaceutically acceptable salt thereof;
said fixed dose combination for use in the treatment of a depressive disorder, including MDD;
a pharmaceutical composition comprising a pharmaceutical carrier and a fixed-dose combination of domperidone and pramipexole; and
a method for treating a depressive disorder, including MDD, in a patient in need of said treatment which comprises administering to said patient said medicament as a fixed-dose combination.

For this method (or use), the invention provides a medicament consisting of or comprising a pharmaceutical composition in dosage unit form which comprises
(a) domperidone, in an amount per unit form equivalent to from 2 mg to 120 mg of domperidone base; and
(b) pramipexole or a pharmaceutically acceptable salt thereof, in an amount equivalent to from 0.125 mg to 45 mg of pramipexole dihydrochloride monohydrate,
   in admixture with a pharmaceutical carrier or vehicle.

According to this fourth aspect of the invention, domperidone as described in “The Domperidone Component (a)” section may be used as Component (a) of said pharmaceutical composition, in an amount per unit form as described in said section, in a fixed dose combination with pramipexole Component (b) in an amount per unit form as described above in “The pramipexole Component (b)” section, in admixture with a pharmaceutical carrier or vehicle.

For this method (or use), the invention provides a fixed-dose combination consisting of or comprising
domperidone, in an amount equivalent to from 2 mg to 120 mg of domperidone base; and
pramipexole or a pharmaceutically acceptable salt thereof, in an amount equivalent to from 0.125 mg to 45 mg of pramipexole dihydrochloride monohydrate.

For its use in the treatment of a depressive disorder, including MDD, in a patient in need of said treatment, said fixed-dose combination is in a pharmaceutical composition comprising a pharmaceutical carrier or vehicle and said fixed-dose combination.

According to this embodiment, domperidone as described in “The Domperidone Component (a)” section may be used in a pharmaceutical composition, in an amount per unit form as described in said section, and pramipexole as described above in “The pramipexole Component (b)” section may be used in pharmaceutical composition (b) in an amount per unit form as described therein.

In particular, said domperidone Component (a) is selected from the group consisting of domperidone base, domperidone hydrochloride, domperidone maleate and domperidone succinate (1:1), in an amount per unit form equivalent to from 2 mg to 120 mg of domperidone base; and said pramipexole Component (b) or pramipexole in pharmaceutical composition (b) is in an amount per unit form equivalent to a range selected from the group consisting of from 0.125 mg to 45 mg, from more than 4.5 mg to 45 mg, from more than 6 mg to 45 mg, from 10 mg to 45 mg, from 13 mg to 45 mg, and from 15 mg to 45 mg, normally from 7.5 mg to 25 mg, from 15 mg to 25 mg, from more than 20 mg to 25 mg and from 20.25 mg to 25 mg of pramipexole dihydrochloride monohydrate.

In pharmaceutical compositions described herein, the domperidone may be in an IR-unit form or ER-unit form and said pramipexole may also be in an IR-unit form or in an ER-unit form. Said unit forms are described in “The formulations” section below.

According to a first preferred embodiment, the invention provides a pharmaceutical composition in dosage unit form comprising
(a) domperidone, in an amount per unit form equivalent to from 2 mg to 60 mg of domperidone base; and
(b) pramipexole or a pharmaceutically acceptable salt or solvate thereof, in an amount per unit form equivalent to from 0.125 mg to 22.5 mg,
   in admixture with a pharmaceutical carrier or vehicle in an IR-formulation.

This composition may be for use for the treatment of depressive disorder, including MDD, in a patient and normally administered to said patient twice a day.

According to this embodiment, for said method (or use), in the fixed-dose combination, said domperidone, in an amount equivalent to from 2 mg to 60 mg of domperidone base, and said pramipexole or pharmaceutically acceptable salt or solvate thereof, in an amount per unit form equivalent to from 0.125 mg to 22.5 mg, in particular from 0.125 mg to less than 1.6 mg, from 1.6 mg to 22.5 mg, from more than 4.5 mg to 22.5 mg, or from more than 6 mg to 22.5 mg, preferably from 7.5 mg to 12.5 mg, from more than 10 mg to 12.5 mg or from 10.125 mg to 12.5 mg of pramipexole dihydrochloride monohydrate, are mixed together and formulated in a pharmaceutical composition, in admixture with a pharmaceutical carrier or vehicle, to be administered to the patient suffering from a depressive disorder, including MDD.

According to a second preferred embodiment, said pharmaceutical composition comprises
(a) domperidone or a pharmaceutically acceptable salt or solvate thereof, in an amount per unit form equivalent to from 4 mg to 120 mg of domperidone base; and
(b) pramipexole or a pharmaceutically acceptable salt or solvate thereof, in an amount per unit form equivalent to from 0.375 mg to 45 mg of pramipexole dihydrochloride monohydrate,
   in admixture with a pharmaceutical carrier or vehicle in an ER-formulation.

This composition is useful or for use for the treatment of depressive disorders, including MDD, in a patient and normally administered to said patient once a day.

According to this embodiment, for said method (or use), in this composition in ER-formulation said domperidone, in an amount equivalent to from 4 mg to 120 mg of domperidone base, and said pramipexole or pharmaceutically acceptable salt or solvate thereof, in an amount per unit form equivalent to from 0.375 mg to 45 mg, in particular from 0.375 mg to less than 1.6 mg, from 1.6 mg to 45 mg, from more than 4.5 mg to 45 mg or from more than 6 mg to 45 mg, normally from 15 mg to 25 mg, from more than 20 mg to 25 mg, from 20.25 mg to 25 mg, or from 15 mg to 22.5 mg, from more than 20 mg to 22.5 mg and from 20.25 mg to 22.5 mg, of pramipexole dihydrochloride monohydrate, are mixed together and formulated in a pharmaceutical composition, in admixture with a pharmaceutical carrier or vehicle, to be administered once a day to the patient suffering from a depressive disorder, including MDD.

The Formulations

For the intended use in the treatment of a depressive disorder, including MDD, in combination with pramipexole, the domperidone Component (a) is formulated in a pharmaceutical composition, wherein said domperidone is in admixture with a pharmaceutical carrier or vehicle. For said treatment, also pramipexole, Component (b) is formulated in a pharmaceutical composition, wherein said pramipexole, is in admixture with a pharmaceutical carrier or vehicle.

The dosage, i.e. the amount of active ingredient in a single dose (amount per unit form) to be administered to the patient, can vary widely depending on the age, weight, and the health condition of the patient. This dosage includes the administration of a domperidone amount from 2 mg to 120 mg, according to the potency the age of the patient, an effective pramipexole amount equivalent to from 0.125 mg to 45 mg of pramipexole dihydrochloride monohydrate, according to the age of the patient, from one to three times a day by intravenous, subcutaneous, oral, or transcutaneous administration, according to the strength of the doses of the each of the active ingredients.

The above pharmaceutical compositions are formulated in admixture with a pharmaceutical carrier or vehicle for any administration route. For example, said pharmaceutical compositions are in a pharmaceutical dosage unit form for oral, intravenous (including infusion), intramuscular, intranasal, intraperitoneal, subcutaneous, transdermal, or rectal administration.

These unit forms are manufactured according to conventional technologies. Particularly advantageous are the formulations in the form of tablets, multi-score tablets, multi-layer tablets, coated tables, orally disintegrating tablets, extended release tablets, hard or soft capsules, multi-compartment capsules, extended-release capsules, suppositories for rectal administration, patches for transdermal administration, liquid oral solutions, syrups or suspensions in a predetermined unit form, apparatus for intravenous infusion, and vials for the intravenous or subcutaneous administration.

The pharmaceutical compositions may be formulated in oral unit forms such as tablets or gelatin capsules wherein the domperidone Component (a), the pramipexole Component (b), or the Component (ab) active ingredients are in admixture with a carrier or vehicle that may include a diluent, such as cellulose, dextrose, lactose, mannitol, sorbitol or sucrose; a lubricant, such as, acid, calcium or magnesium stearate, polyethylene glycol, silica, or talc; and if needed, a binder such as magnesium aluminum silicate, gelatin, methylcellulose, sodium carboxymethylcellulose, or polyvinylpyrrolidone.

Said oral unit forms may be tablets coated with sucrose or with various polymers for an immediate release. Alternatively, the tablets can be manufactured by using carriers such as acrylic and methacrylic acid polymers and copolymers; cellulose derivatives such as hydroxypropylethylcellulose; or other appropriate materials having a prolonged or delayed activity by progressively releasing a predetermined quantity of active ingredient. For example, the unit forms may be formulated in tablets in which Component (b) is in ER-formulation, for example in admixture with hydroxypropyl methyl cellulose or in a film-coated microgranule. Carriers and vehicles for ER-tablets include retardant materials such as acrylic and methacrylic acid polymers and copolymers; the aforementioned cellulose derivatives such as hydroxypropylmethylcellulose, hydroxyethylcellulose, hydroxypropylethylcellulose, hydroxypropylcellulose, methylcellulose, ethylcellulose, or sodium carboxymethylcellulose; gums; waxes; glycerides or aliphatic alcohols or a mixture thereof.

Syrups and orally dispersible tablets may also comprise sweeteners, lubricants, taste-masking agents, binders, and coloring agents.

Suppositories are manufactured by using a suppository base such as cocoa butter, poloxamers combined with solvents such as. polyethylene glycols (for example PEG 3350), propylene glycol, or triglycerides according to conventional technologies.

A Transdermal drug delivery system (TDDS) provides transdermal delivery using transdermal drug formulations and transdermal patches incorporating such transdermal drug formulations. A transdermal drug delivery system may include a composition in form of a patch, a cream, a gel, a lotion or a paste comprising for example a domperidone, pramipexole or both the active ingredients.

A typical TDDS is a patch formulation wherein the active ingredient or the mixture of the active ingredients may comprise adjuvants such as D-sorbitol, gelatin, kaolin, methyl paraben, polysorbate 80, propylene glycol, propyl paraben, povidone, sodium carboxymethylcellulose, sodium polyacrylate, tartaric acid, titanium dioxide, and purified water. A patch formulation may also contain skin permeability enhancer such as lactate esters (e.g., lauryl lactate), triacetin or diethylene glycol monoethyl ether.

Unit forms may be formulated in tablets in which Component (a) and Component (b) each in ER-formulation, for example domperidone base and pramipexole dihydrochloride monohydrate, each in admixture with hydroxypropyl methyl cellulose or in a film-coated microgranule. These unit forms (a) and (b) are destined to be concurrently or sequentially administered to a patient suffering from a depressive disorder, including MDD in combination with an oral unit form such as a tablet or gelatin capsule wherein Component (a) is formulated with a diluent and a lubricant in an IR-formulation, or in a tablet or capsule for extended release.

Domperidone may be formulated in a pharmaceutical composition, wherein said domperidone is in admixture with a pharmaceutical carrier or vehicle. Pramipexole may also be formulated in a pharmaceutical composition, wherein said pramipexole is in admixture with a pharmaceutical carrier or vehicle.

In the above combinations, including fixed-dose combinations, of the present invention, the dose of domperidone or pharmaceutically acceptable salt or solvate thereof, in domperidone base, per unit form, is from 2 mg to 120 mg, in an IR- or ER-formulation.

### Example 1

The ability of domperidone to prevent the gastro-intestinal (GI) adverse effects (AEs) of pramipexole in humans was tested.

A Phase I study was conducted in subjects receiving a single oral dose of pramipexole dihydrochloride monohydrate (“pramipexole”) with or without a single oral dose of domperidone base (“domperidone”). The study was a single center study.

The objective of the study was to demonstrate that domperidone could safely attenuate the gastro-intestinal side effects of pramipexole given in doses equivalent or higher than those approved in the treatment of Parkinson's Disease or shown in clinical trials to be marginally effective in the treatment of depression.

To be enrolled in the study, participants the following inclusion/exclusion key criteria:

### Key Inclusion Criteria

1. Male and female subjects aged 20-45 years old both ages included.
2. Females of childbearing potential must agree to be abstinent or else use any two of the following medically acceptable forms of contraception from the Screening Period through 14 days after the study Exit Visit: condom with spermicidal jelly, diaphragm or cervical cap with spermicidal jelly, or intrauterine device (IUD). A female whose male partner has had a vasectomy must agree to use one additional form of medically acceptable contraception. Subjects must agree to practice the above birth control methods for 14 days after the final visit as a safety precaution.
3. Females of non-childbearing potential, defined as surgically sterile (status post-hysterectomy, bilateral oophorectomy, or bilateral tubal ligation) or post-menopausal for at least 12 months, do not require contraception during the study. The reason must be documented in the source documents.
4. Males with female partners of childbearing potential must agree to use a highly effective, medically acceptable form of contraception from the Screening Period through 14 days after the study Exit Visit. Males with female partners of childbearing potential who themselves are surgically sterile (status post vasectomy) must agree to use condoms with spermicide over the same period of time. Male subjects must agree to practice the above birth control methods for 14 days after the final visit as a safety precaution.
5. Subjects must be in good health as determined by their medical history including personal and family psychiatric history and results of physical examination, electrocardiogram (ECG), vital signs, and laboratory tests. A subject with a medical abnormality may be included only if the investigator or designee considers that the abnormality will not introduce significant additional risk to the subject's health or interfere with study objectives. 6. Subjects must be able to clearly and reliably communicate changes in their medical condition.
7. Subjects with a body mass index (BMI) between 19.0 and 32.0 kg/m² (both inclusive).
8. Subjects able to swallow multiple pills or capsules simultaneously.
9. Subjects must have signed an informed consent form indicating that they understand the purpose of and procedures required for the study and are willing to participate in the study and comply with the study procedures and restrictions.

### Key Exclusion Criteria:

The criteria for exclusion of a subject from enrollment in the study were as follows:
1. Any clinically relevant acute or chronic diseases which could interfere with the subjects' safety during the trial, expose them to undue risk, or interfere with the study objectives.
2. History or presence of gastrointestinal, hepatic, or renal disease or other condition known to interfere with the absorption, distribution, metabolism or excretion of the study drugs.
3. History of substance abuse, known drug addiction, or positive test for drugs of abuse or alcohol.
4. History of drug or other significant allergy.
5. Known hypersensitivity to pramipexole, or to domperidone or similar dopamine receptor antagonists.
5. History of and/or current QT interval prolongation, congenital long QT syndrome, electrolyte abnormalities (e.g., hypokalemia or hypomagnesemia), congestive heart failure, bradyarrhythmias or other medicinal products that lead to QT prolongation or 1st degree AV block at Screening, Day −1, or pre-dose, ≥450 QTcF for males and ≥470 QTcF for females.
7. Treatment with centrally active drugs or antiemetics, within 1 months of study entry.
8. Tobacco or nicotine users (except subjects who stopped using tobacco or nicotine 1 year or more before enrollment in the study).
9. Excessive daily consumption of xanthines containing drinks (i.e. >500 mg/day of caffeine).
10. Subjects unwilling to curtail prolonged intensive physical exercise during the study conduct (from the Screening visit until the last dose of study drug).
11. Positive test result for hepatitis B surface antigen, hepatitis C antibody.
12. Positive test result for HIV 1 or 2 serology.
13. Likely to need any medical or dental treatment during the study period.
14. Use of any prescription or over-the-counter medication within 14 days prior to admission on Day-1. In addition, any medications with central effects are prohibited for a period equal to 5 times the drug half-life prior to admission (Day −1), should this period be longer than 14 days.
15. Subjects unlikely to co-operate during the study, and/or be questionably compliant in the opinion of the investigator.
16. Subjects unable to be contacted in case of an emergency.
17. Intake of an investigational drug within 30 days of study entry.
18. Show evidence of suicidal ideation within the last 6 months as assessed by the C-SSRS (Columbia Suicide Severity Rating Scale) at Screening.

Following enrollment in the study, participants received single increasing oral doses of pramipexole given once daily in the morning (Period 1 of the study). The starting dose of pramipexole was 0.5 mg and the dose was increased daily by 0.5 mg increments. Once a subject had reached his/her first intolerable dose (FID-1), upward dose escalation was discontinued. First intolerable dose (FID) was defined as:
one (1) episode of vomiting; or
Two (2) episodes of retching, or
One (1) episode of severe nausea (Grade 3; defined as nausea interfering with activities of daily living or inadequate oral caloric or fluid intake; tube feeding, total parenteral nutrition or hospitalization indicated) lasting more than 1 hour, or
Three (3) consecutive episodes at every 4 hour ratings of moderate nausea (Grade 2; defined as subjectively symptomatic, but not interfering with activities of daily living), or
One (1) episode of moderate diarrhea (Grade 2; defined as 4-6 stools more than at baseline).

When a subject reached FID-1 on pramipexole alone, the subject was washed out for at least 5 days, and then entered Period 2 of the study during which the subject received single daily oral doses of pramipexole starting at 0.5 mg and titrated upward by 0.5 mg increments, together with oral domperidone (5 mg) until subjects again reached an intolerable dose defined as above. The FID on oral pramipexole plus oral domperidone was referred to as FID-2.

If a subject reached FID-2 during Period 2 at the same or lower dose than FID-1, and providing the investigator judged there were no safety issues and the subject was consenting, the subject received the same dose of pramipexole as the FID-2 dose together with a higher dose of oral domperidone (10 mg) on the next day and the protocol specified that said subject should continue with the remainder of the dose titration with the higher dose of oral domperidone (10 mg) until they reach the intolerable dose (FID2+). All other provisions of the protocol remained unchanged. Assessments were the same as those planned for the dose escalation day.

On each study day, subjects were followed up for up to 8 hours following drug administration for AEs, vital signs, ECGs. In addition, a laboratory panel was taken at screening and at the end of the study.

All subjects reached FID-1 (pramipexole alone) during the study. The dose limiting toxicity was gastro-intestinal adverse events in all subjects. For all subjects FID-2 was higher than FID-1. Results showed that concomitant administration of domperidone with pramipexole prevented the occurrence of dose-limiting gastro-intestinal adverse events associated with high doses of pramipexole.

Taken together, results showed that the co-administration of domperidone with pramipexole attenuated dose-limiting gastro-intestinal adverse effects reported with pramipexole alone, thus showing that a domperidone enables the administration to a human being of pramipexole in doses otherwise non-tolerated when administering pramipexole alone.

### REFERENCES

Barone et al. 2010: Barone P, Poewe W, Albrecht S, Debieuvre C, Massey D, Rascol O, Tolosa E, Weintraub D. “Pramipexole for the treatment of depressive symptoms in patients with Parkinson's disease: a randomised, double-blind, placebo-controlled trial.” Lancet Neurol. 2010 June; 9(6):573-80. doi: 10.1016/S1474-4422(10)70106-X. Epub 2010 May 7.
Brogden et al 1982: Brogden R N, Carmine A A, Heel R C, Speight T M, Avery G S. “Domperidone. A Review of its Pharmacological Activity, Pharmacokinetics and Therapeutic Efficacy in the Symptomatic Treatment of Chronic Dyspepsia and as an Antiemetic”. Drugs 1982 (November) 24(5):360-400.
Bruni et al 2013: Bruni G, Maietta M, Scotti F, Maggi L, Bini M, Ferrari S, Capsoni D, Boiocchi M, Berbenni V, Milanese C, Girella A, Marini A. “Structure and properties of domperidone and its succinate salt” Acta Crystallogr B Struct Sci Cryst Eng Mater. 2013 August; 69(Pt 4):362-70. doi: 10.1107/S2052519213016989. Epub 2013 Jul. 18.
Corrigan et al. 2000: Corrigan M H, Denahan A Q, Wright C E, Ragual R J, Evans D L; Corrigan M H, Denahan A Q, Wright C E, Ragual R J, Evans D; “Comparison of pramipexole, fluoxetine, and placebo in patients with major depression”; Depress Anxiety. 2000; 11(2):58-65.
Cusin et al. 2013: Cusin C, Iovieno N, Iosifescu D V, Nierenberg A A, Fava M, Rush A J, Perlis R H. A randomized, double-blind, placebo-controlled trial of pramipexole augmentation in treatment-resistant major depressive disorder. J Clin Psychiatry. 2013 July; 74 (7).
Dell'Osso et al 2013: Dell'Osso B, Ketter T A. Assessing efficacy/effectiveness and safety/tolerability profiles of adjunctive pramipexole in bipolar depression: acute versus long-term data. Int Clin Psychopharmacol. 2013 November; 28(6):297-304.
de Souza et al 2013: de Sousa R T, Zanetti M V, Brunoni A R, Machado-Vieira R. Challenging Treatment-Resistant Major Depressive Disorder: A Roadmap for Improved Therapeutics. Current Neuropharmacology, 2015, 13, 616-635.
Fawcett et al 2016: Fawcett J, Rush A J, Vukelich J, Diaz S H, DuNKllee L, Romo P, Yarns B C, Escalona R, Clinical Experience With High-Dosage Pramipexole in Patients With Treatment-Resistant Depressive Episodes in Unipolar and Bipolar Depression. Am J Psychiatry. 2016 Feb. 1; 173(2):107-11.
Goldberg et al 2004: Goldberg J F, Burdick K E, Endick C J. Preliminary randomized, double-blind, placebo-controlled trial of pramipexole added to mood stabilizers for treatment-resistant bipolar depression. Am J Psychiatry. 2004 March; 161(3): 564-6.
Hon et al. 2012: Hori H, Kunugi H. The efficacy of pramipexole, a dopamine receptor agonist, as an adjunctive treatment in treatment-resistant depression: an open-label trial. Scientific World Journal. 2012; 2012:372474.
Kleeblatt et al 207: Kleeblatt J, Betzler F, Kilarski L L, Bschor T, Köhler S. Efficacy of off-label augmentation in unipolar depression: A systematic review of the evidence. Eur Neuropsychopharmacol. 2017 Mar. 16. pii: 50924-977X(17)30185-2.
Latha et al. 2012: Latha S, Selvamani P, Lakshmana Prabu S, Santhosh Kumar P, Pal T K. “Formulation and Evaluation of Matrix Diffusion Controlled Transdermal Patches of Domperidone hydrochloride”. Ars Pharm. 2011; 52(1): 31-36.
Madishetti et al 2010: Madishetti S K, Palem C R, Gannu R., Thatipamula R P, Panakanti P K, and Yamsani M R; “Development of domperidone bilayered matrix type transdermal patches: physicochemical, in vitro and ex vivo characterization”; Daru 2010; 18(3): 221-229.
Piercey 1998: Piercey M F. Pharmacology of pramipexole, a dopamine D3 preferring agonist, useful in treating Parkinson's disease. Clin Neuropharmacol 1998; 22.5:141-151,
Poon et al. 2013: Poon S, Sim K, Baldessarini R J. Pharmacological Approaches for Treatment-resistant Bipolar Disorder. Curr Neuropharmacol. 2015; 13(5):592 604.
Prabhu et al 2011: Prabhu P, Shah 5, Gundad S, “Formulation development and investigation of domperidone transdermal patches”. Int J Pharm Investig. 2011 October-December; 1(4):240-246.
Samuels et al. 2007: Samuels E R, Hou R H, Langley R W, Szabadi E, Bradshaw C M. “Comparison of pramipexole with and without domperidone co-administration on alertness, autonomic, and endocrine functions in healthy volunteers”. Br J Clin Pharmacol. 2007 November; 64(5):591-602.
Shirisha et al. 2017: Shirisha S, Joshi G, Sahoo G K, Rao Y M; “Preparation and Evaluation of Matrix Type Transdermal Patches of Domperidone Maleate: in vitro and ex vivo Characterization”; Indian J of Pharmaceutical Education and Research. 2017; 51(4):517-24.
Schneider C S and Mierau J, 1987: Schneider C S, Mierau J “Dopamine autoreceptor agonists: resolution and pharmacological activity of 2,6-diaminotetrahydrobenzothiazole and an aminothiazole analogue of apomorphine”. J. Med Chem. 1987 March; 30(3):494-8.
Sienaert et al 2013: Sienaert P, Lambrichts L, Dols A, De Fruyt J. Evidence-based treatment strategies for treatment-resistant bipolar depression: a systematic review. Bipolar Disord. 2013 February; 15(1):61-9.
Tondo et al 2014: Tondo L, Vázquez G H, Baldessarini R J. Options for pharmacological treatment of refractory bipolar depression. Curr Psychiatry Rep. 2014 February; 16(2):431.
Willner et al. 1994: Willner P, Lappas S, Cheeta S, Muscat R. Reversal of stress induced anhedonia by the dopamine agonist, pramipexole. Psychopharmacol 1994; 115:454-462.

1. Domperidone for use for the treatment of depression in a patient in need of said treatment, in combination with an effective daily dose of (S)-6-propylamino-4,5,6,7-tetrahydro-1,3-benzothiazole-2-amine (pramipexole) or a pharmaceutically acceptable salt or solvate thereof.

2. Domperidone for use according to claim 1, wherein said effective daily dose of (S)-6-propylamino-4,5,6,7-tetrahydro-1,3-benzothiazole-2-amine (pramipexole) or a pharmaceutically acceptable salt or solvate thereof is equivalent to from 0.375 mg to 45 mg of pramipexole dihydrochloride monohydrate.

3. Domperidone, for use according to claim 1, wherein said effective daily dose of (S)-6-propylamino-4,5,6,7-tetrahydro-1,3-benzothiazole-2-amine (pramipexole) or a pharmaceutically acceptable salt or solvate thereof is equivalent to from 15 mg to 25 mg of pramipexole dihydrochloride monohydrate.

4. Domperidone for use according to claim 1, wherein, said domperidone is in a pharmaceutical composition comprising a pharmaceutical carrier and said domperidone, in an amount per unit form equivalent to from 2 mg to 120 mg of domperidone base, in combination with said pramipexole, also in a pharmaceutical composition comprising pramipexole or a pharmaceutically acceptable salt or solvate thereof, in an amount per unit form of from 0.125 mg to 45 mg of pramipexole dihydrochloride monohydrate in admixture with a pharmaceutical carrier or vehicle.

5. Domperidone for use according to claim 1, wherein said domperidone is in a pharmaceutical composition comprising a pharmaceutical carrier or vehicle and a fixed-dose combination of said domperidone or pharmaceutically acceptable salt or solvate thereof, in an amount equivalent to from 2 mg to 120 mg of domperidone base, and of pramipexole or pharmaceutically acceptable salt or solvate thereof, in an amount equivalent to from 0.125 mg to 45 mg of pramipexole dihydrochloride monohydrate.

6. Domperidone for use according to claim 4, wherein said pramipexole or pharmaceutically acceptable salt or solvate thereof is present in said composition in an amount equivalent to from 7.5 mg to 25 mg of pramipexole dihydrochloride monohydrate.

7. Domperidone for use according to claim 4, wherein any composition is in dosage unit form.

8. A pharmaceutical composition for use in the treatment of depression comprising a pharmaceutically acceptable carrier or vehicle and a fixed dose combination of domperidone or a pharmaceutically acceptable salt or solvate thereof and pramipexole or a pharmaceutically acceptable salt or solvate thereof.

9. The composition of claim 8, wherein said domperidone or pharmaceutically acceptable salt or solvate thereof is present in an amount equivalent to from 2 mg to 120 mg of domperidone base and said pramipexole or pharmaceutically acceptable salt or solvate thereof is present in an amount equivalent to from 0.125 mg to 45 mg of pramipexole dihydrochloride monohydrate.

10. The composition of claim 8, wherein said carrier or vehicle is for an IR-formulation; and, in said fixed-dose combination,
said domperidone or pharmaceutically acceptable salt or solvate thereof is present in an amount equivalent to from 2 mg to 60 mg of domperidone base; and
said pramipexole or pharmaceutically acceptable salt or solvate thereof is present in an amount-range, in pramipexole dihydrochloride monohydrate, selected from the group consisting of from 7.5 mg to 22.5 mg and from 7.5 to 12.5 mg.

11. The composition of claim 8, wherein said carrier or vehicle is for an ER-formulation; and, in said fixed-dose combination,
said domperidone or pharmaceutically acceptable salt or solvate thereof is present in an amount equivalent to from 4 mg to 120 mg of domperidone base; and
said pramipexole or pharmaceutically acceptable salt or solvate thereof is present in an amount-range, in pramipexole dihydrochloride monohydrate, selected from the group consisting of from 15 mg to 45 mg and from 15 mg to 25 mg.

12. The composition according to claim 9, which is in dosage unit form.

13. A pharmaceutical composition comprising
(a) domperidone or a pharmaceutically acceptable salt or solvate thereof, in an amount equivalent to from 2 mg to 120 mg of domperidone base; and
(b) pramipexole or a pharmaceutically acceptable salt or solvate thereof, in an amount equivalent to from 0.125 mg to 45 mg of pramipexole dihydrochloride monohydrate,
in admixture with a pharmaceutical carrier or vehicle.

14. The composition of claim 13, which is in dosage unit form.

15. Use of domperidone for the preparation of a medicament for the treatment of depression in combination with pramipexole or a pharmaceutically acceptable salt or solvate thereof.

16. A method for the treatment of a patient suffering from depression, which comprises administering to said patient domperidone, at a daily dose (in domperidone base) of from 4 mg to 120 mg, in combination with pramipexole, at a daily dose (in pramipexole dihydrochloride monohydrate) of from 0.375 mg to 45 mg.

17. The method of claim 16 wherein said pramipexole daily dose (in pramipexole dihydrochloride monohydrate) is from 15 mg to 25 mg.
